(19) United States
(12) Patent Application Publication
(10) Pub. No.: US 2005/0249668 A1
(43) Pub. Date: Nov. 10, 2005

(54) Nir-fluorescent cyanine dyes, their synthesis and biological use

(76) Inventors: Ralph Weissleder, Peabody, MA (US); Ching-Shuan Tung, Wayland, MA (US); Yuhui Lin, Northborough, MA (US)

Correspondence Address: FISH & RICHARDSON PC, P.O. BOX 1022, MINNEAPOLIS, MN, 55440-1022, US

(21) Appl. No.: 10/496,239

(22) PCT Filed: Mar. 31, 2003
(86) PCT No.: PCT/US03/09879

§ 371 Date: Jul. 5, 2005

Related U.S. Application Data
(60) Provisional application No. 60/368,962, filed on Mar. 29, 2002.

(51) Int. Cl. A61K 49/00; C07D 413/02; C07D 417/02; C07D 43/02; C07K 14/47

(52) U.S. Cl. 424/9.6; 530/409; 548/150; 548/217; 548/302.1

(57) ABSTRACT

The invention includes new water-soluble NIR fluorochromes, e.g., for biomedical imaging. The new dyes are highly stable, asymmetric cyanine compounds, characterized by 1) superior chemical stability, 2) excellent optical properties (e.g., high quantum yield), 3) bio-compatibility, 4) conjugatability and 5) ideal in vivo imaging properties. Monoactivated hydroxysuccinimide esters of the new dyes are highly reactive with peptides, metabolites, proteins, peptide-folate conjugates, and other biological macromolecules and affinity ligands, forming stable complexes. Affinity molecules tagged with the new dyes can be used, for example, for imaging of tumors in vivo.

### CROSS REFERENCE TO RELATED APPLICATION

This application claims the benefit of the filing date of U.S. Provisional Application Ser. No. 60/368,962, filed on Mar. 29, 2002. The contents of this application is hereby incorporated herein by reference in its entirety.

### FIELD OF THE INVENTION

This invention relates to chromophores for optical imaging, and more particularly to asymmetric near infrared (NIR) chromophores and methods for their synthesis and use.

### BACKGROUND OF THE INVENTION

Light-based imaging methods provide a non-invasive avenue for extracting biological information from living subjects. These methods measure various native parameters of tissues through which photons can travel. Such parameters include absorption, scattering, polarization, spectral characteristics, and fluorescence. While light in the visible range (i.e., 400-650 nm) can be used for analysis of tissue surface structures and intravital microscopy of relatively shallow tissues (i.e., less than about 800 μm below the tissue surface), imaging of deeper tissues generally requires the use of near infrared (NIR) light. NIR radiation (approx. 600-1000 nm) exhibits tissue penetration of up to ten centimeters, and can accordingly be used for imaging internal tissues (see, e.g., Wyatt, Phil. Trans. R. Soc. London B, 352:701-706, 1997; and Tromberg et al., Phil. Trans. R. Soc. London B. 352:661-667, 1997).

Besides being non-invasive, NIR fluorescence imaging methods offer a number of advantages over other imaging methods: they provide generally high sensitivity, do not require exposure of test subjects or lab personnel to ionizing radiation, can allow for simultaneous use of multiple, distinguishable probes (important in molecular imaging), and offer high temporal and spatial resolution (important in functional imaging and in vivo microscopy, respectively).

In NIR fluorescence imaging, filtered light or a laser with a defined bandwidth is used as a source of excitation light. The excitation light travels through body tissues. When it encounters an NIR fluorescent molecule (i.e., a “contrast agent”), the excitation light is absorbed. The fluorescent molecule then emits light that has, for example, detectably different spectral properties (e.g., a slightly longer wavelength) from the excitation light. Despite good penetration of biological tissues by NIR light, conventional NIR fluorescence probes are subject to many of the same limitations encountered with other contrast agents, including low target/background ratios.

A number of reflectance and tomographic imaging systems have recently been developed to detect NIR fluorescence in deep tissues, including in patients (Ntziachristos et al., Proc. Natl. Acad. Sci. USA., 97:2767-72 (2000)). Nonetheless, there is a need for a new generation of biocompatible fluorochromes. Indocyanine green (ICG) has been used clinically for over 20 years with few side effects (Hope-Ross et al., Ophthalmology, 101:529-533 (1994)), but its use in designing targeted agents is limited by the fact that monoderivatized activated precursors are not available. Moreover, ICG is hydrophobic and exhibits a high degree of albumin binding and nonlinear fluorescence. Synthetic fluorochromes have been plagued by problems such as significant spectral broadening as wavelengths increase, low quantum yield, photoinstability, chemical instability with increasing red-shift, and a tendency to aggregate as a result of large planar surfaces and/or hydrophobicity.

### SUMMARY OF THE INVENTION

The invention is based, in part, on the discovery and synthesis of new water-soluble NIR chromophores for biomedical imaging. The new chromophores are highly stable, asymmetric cyanine compounds, characterized by 1) superior chemical stability, 2) excellent optical properties (e.g., high quantum yield), 3) bio-compatibility, 4) conjugatability, and 5) ideal in vivo imaging properties. Monoactivated hydroxysuccinimide esters of the new chromophores are highly reactive with peptides, metabolites, proteins, peptide-folate conjugates, and other biological macromolecules and affinity ligands, forming stable complexes that can be used as biocompatible probes. Affinity molecules tagged with the new chromophores can be used, for example, for imaging of tumors in vivo.

In one aspect, the invention features asymmetrical chromophores having the following formula:

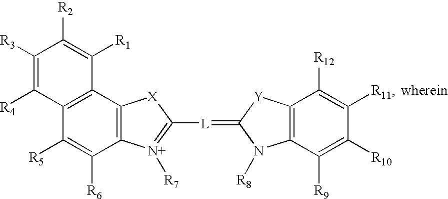

L is a conjugated linker moiety (e.g., L can be (CH═CH—)ₙCH, where n=1,2, 3, or 4, or can include one or more conjugated ring structures).

R-₁₋₁₂ can be, independently, hydrogen, substituted or unsubstituted alkyl groups (where substituted means that one or more hydrogen atoms are replaced by carbon-, nitrogen-, oxygen-, phosphorus-, and/or hydrogen-containing functional groups), substituted or unsubstituted alkenyl groups, substituted or unsubstituted alkynyl groups, substituted or unsubstituted aryl groups, sulfur-containing functional groups, phosphorus-containing functional groups, oxygen-containing functional groups, or nitrogen-containing functional groups; and

X and Y can be the same or different, and can be, for example, —O—, —S—, —NH— (or a substituted variant thereof where H is replaced by an alkyl, alkenyl, alkynyl, aryl, or other moiety), or substituted or unsubstituted methylene (—CH₂). Thus, for example, X and Y can both be dimethylmethylene groups (i.e., —C(CH₃)₂—).

One or more of R₁₋₁₂ can include a reactive group for conjugation to a macromolecule (e.g., an amino group for conjugation with an carboxylate derivative, or vice versa) to form a molecular probe (e.g., an imaging probe). In some cases, one or more of R-₁₋₁₂ can include at least one sulfate, sulfonate, phosphate, phosphonate, halide, nitro, nitrile, nitrate, or carboxylate group. In particular embodiments, for example, R₁, R₃, R₅, R₆, R₉, R₁₀, and R₁₂ can all be hydrogen, R₂ and R₄ can both be —₃⁻, R₇ can be —CH₂CH₃, R₈ can be (CH₂)₄SO₃⁻, R₁₁ can be CO₂H, and X and Y can be —C(CH₃)₂—.

In another aspect, the invention features asymmetrical chromophores having the formula: 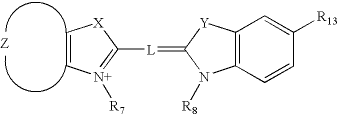
where L, R₇, R₈, X, and Y are defined as above; R₁₃ is C(O)OR₁₄ or NHC(O)CH₂J; R₁₄ is H or

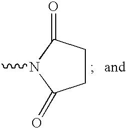

Z is a group of nonmetallic atoms necessary for forming a substituted or unsubstituted, condensed aromatic ring or ring system. Thus, for example, Z can be either of: 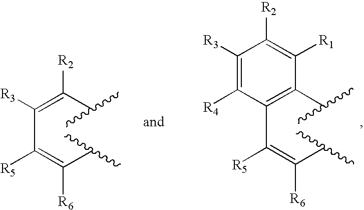
where R₂₋₆ are defined as above.

In the case where Z is 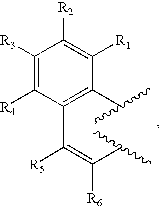
R₁, R₃, R₅, and R₆ can be hydrogen, R2 and R4 can be —SO₃⁻, can be —CH₂CH₃, R₈ can be (CH₂)₄SO₃⁻, and X and Y can be —C(CH₃)₂—.

In the case where Z is

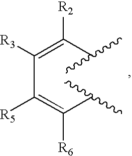

R₂, R₅, and R₆ can be hydrogen, R₃ can be —SO₃⁻, R₇ can be —CH₂CH₃, R₈ can be (CH₂)₄SO₃⁻, and X and Y can be —C(CH₃)₂.

In a further aspect, the invention features asymmetrical chromophores having the formula: 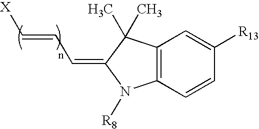
where X is selected from the group consisting of:

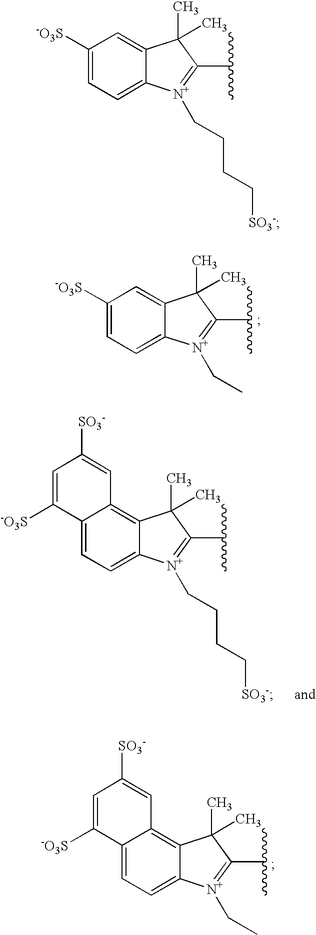

R₈, R₁₃ and R₁₄ are defined as above and n=2 or 3.

Embodiments can include one or more of the following.

R₁₄ can be H

J can be Cl or I. R₈ can be CH₃ or (CH₂)₄SO₃⁻.

The invention also features molecular and/or imaging probes that include the new chromophores.

In another aspect, the invention features methods of gene sequence recognition using fluorescently labeled nucleic acid recognition molecules, including DNA, RNA, modified nucleic acid, PNA molecular beacon, or other nucleic acid binding molecules. The methods include the use of one or more of the chromophores described above, together with any one or combination of well-known techniques such as hybridization, ligation, cleavage, recombination, synthesis, sequencing, mutation detection, real-time polymerase chain reactions, in situ hybridization, and the use of microarrays.

The invention also features in vivo imaging methods (e.g., NIR imaging in a human or animal) for imaging tissue (e.g., a living tissue, e.g., a diseased tissue). The methods include a) conjugating to a targeting ligand (e.g., an antibody, a protein, a peptide, a receptor binding ligand, a small ligand, or a carbohydrate) a chromophore as described above; b) combining the conjugated chromophore with a suitable excipient to form an injectable or otherwise administerable formulation; c) administering the formulation to a tissue; and d) detecting the conjugated chromophore (e.g., by using NIR spectroscopy) in the tissue to provide a fluorescence image of the tissue. The imaging method can be used, for example, in the detection of disease (e.g., cancer, CNS diseases, cardovascular diseases, arthritis) at an early stage or at the molecular level; for characterization of disease sensitivity, prognosis, and/or molecular profile; or for determination of drug efficacy at the molecular level, or response to particular drugs, to optimize drug dosage in individual patients, or for drug discovery in vivo. The same methods can be used for in vitro imaging, although, in that case, the combining with an excipient and administering steps can generally be omitted.

The invention also features in vivo enzyme sensing methods. The methods include a) conjugating, to an enzyme-activatable molecule, a chromophore as described above; b) combining the conjugated chromophore with a suitable excipient to form an injectable formulation; c) injecting the formulation into a tissue (e.g., so that the injected chromophore will interact with specific enzymes and cause optical signal changes); and d) detecting the conjugated chromophore in the tissue to provide information about targeted enzymes. As above, the same method can be used for in vitro enzyme sensing (e.g., without the combination and injection steps).

The chromophores described above can also be used as free dyes for in vivo imaging.

The fluorescence signal generated by the chromophores described above, or conjugates thereof, whether collected by tomographic, reflectance, endoscopic, video imaging technologies, or other methods, and whether used quantitatively or qualitatively, is also considered to be an aspect of the invention.

As used herein, the terms “fluorochrome” and “fluorochrome dye” both refer to chromophores that are able to absorb energy at a ground state and emit fluorescence light from an excited state. The chromophores can be conjugated with other molecules (e.g., biological macromolecules) to form molecular probes (e.g., imaging probes, e.g., NIR fluorescence probes).

As used herein, the term “asymmetrical chromophore” refers to a chromophore of formula A-L-B, where A and B are non-identical unsaturated moieties, and L is a linker that includes conjugate double bonds.

The invention provides several advantages. For example, the new chromophores offer: 1) peak fluorescence in or close to the 700-900 nm range, which is ideal for optical in vivo imaging, 2) high quantum yield, 3) narrow excitation/emission spectra, 4) high chemical- and photo-stability, 5) low or no toxicity, 6) water-solubility, 7) biocompatibility, biodegradability, and excretability, 8) availability of monofunctional derivatives as a platform technology, and 9) commercial viability and scalability of production for large quantities required for human use. Moreover, the new chromophores are asymmetric to avoid stacking of large planar surfaces, contain multiple hydrophilic groups, and can be prepared as monohydroxy succinimide esters for binding to biomolecules such as peptides, metabolites, proteins, targeting ligands, DNA and other biomolecules.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and are not intended to be limiting.

Other features and advantages of the invention will be apparent from the following detailed description, and from the claims.

### BRIEF DESCRIPTION OF DRAWINGS

FIG. 1 is a schematic representation of the synthesis of two NIR chromophores of the invention, referred to herein as NIR1 and NIR2.

FIG. 2 is a schematic representation of the synthesis of two NIR chromophores of the invention, referred to herein as NIR3 and NIR4.

FIG. 3A is a schematic representation of the synthesis of four NIR chromophores of the invention, referred to herein as NIR5, NIR6, NIR7, and NIR8

FIG. 3B is a schematic representation of the synthesis of synthetic intermediates 8 and 9, used in the synthesis of NIR7 and NIR8.

FIGS. 4A and 4B are a pair of spectra corresponding to the absorption spectra of NIR1, NIR2, NIR3, and NIR4 (4A) and the fluorescence (excitation and emission) spectra of NIR1 and NIR2 (4B).

FIG. 5A is a schematic representation of the activation of NIR2 with N-hydroxysuccinimide.

FIG. 5B is a set of high performance liquid chromatography (HPLC) traces of NIR2 before (top) and after (bottom) activation.

FIG. 5C is a schematic representation of the conversion of NIR5, NIR6, NIR7, and NIR8 to NIR9, NIR10, NIR11, and NIR12.

FIG. 5D is a set of high performance liquid chromatography (HPLC) traces of of NIR10 and the NIR10-peptide conjugate.

FIG. 5E is a spectrum corresponding to the fluorescence (excitation and emission) spectra of NIR10-peptide conjugate.

FIG. 6 is a digitized photograph showing the fluorescences of NIR1 (well 1), NIR2 (well 2), NIR3 (well 3), NIR4 (well 4), and indocyanine green (IGC; well 5) in response to white light (“light”) and two NIR frequency ranges (i.e., “700 nm” and “800 nm”).

FIG. 7 is a bar graph of the fluorescence intensity (y-axis) of NIR2 attached to a PEGylated graft copolymer having a lysine backbone (i.e., an “NIR2/PGC Probe”) before (white bars) and after (black bars) cleavage by trypsin for 3 hours. The numbers on the x-axis represent the number of NIR2 residues per PCG molecule.

FIG. 8 is a schematic representation of the coupling of a folate-peptide conjugate to NIR2.

FIG. 9 is a digitized photograph of a tumor-bearing mouse, imaged using fluorescence imaging 4 hours after injecting the mouse with folate-derivatized NIR2.

FIG. 10 is a graphical representation corresponding to the cellular uptake of ³H-folate in the KB and HT1080 tumor cell lines.

FIG. 11 is a digitized photograph of KB and HT1080 tumor cells incubated with NIR2-folate probe (0.1 μm) for 30 minutes at 37° C.

FIGS. 12A, 12B, 12C, and 2D are digitized photographs of FR expression and hematoxylin-eosin staining of KB and HT1080 tumors.

FIG. 13A is a digitized photograph of a white light image obtained 24 hours after intravenous injection of the NIR2-folate probe in a representative animal.

FIG. 13B is a digitized photograph of of enlarged NIRF images of the chest tumors.

FIG. 13C is a digitized photograph of of enlarged NIRF images of the low abdomen KB tumors.

FIG. 13D is a digitized photograph of a white light image obtained 24 hours after intravenous injection of the NIR2-folate probe in a representative animal.

FIG. 14 is a graphical representation of the in vivo fluorescence signal of tumors and normal tissues.

FIG. 15 is a graphical representation of time course of KB tumor with various probes.

Unless otherwise noted, like reference symbols in the various drawings indicate like elements.

### DETAILED DESCRIPTION OF THE INVENTION

The invention is directed to highly stable, water-soluble, asymmetric cyanine compounds and their use as chromophores. In general, the new compounds include at least one reactive functional group (e.g., a mono-reactive carboxyl group) that can be used for labeling (i.e., a chromophore attachment moiety). When multiple chromophores are attached to a single macromolecule, fluorescence quenching can be observed. The new biocompatible chromophores, and molecular probes made therefrom, incorporate these properties, and can be used for in vivo detection of specific protease activity, particularly for those proteases that play key roles in different aspects of cancer growth, metastases formation, and angiogenesis (Weissleder et al., Nature Biotech, 17:375-378, 1999; Tung et al., Canc. Res., 2000:4953-4958,2000; Bremer et al., Nat. Med., 7:743-748,2001). The chromophores can, for example, be attached to a partially PEGylated graft copolymer (PGC) with a polylysine backbone (Bogdanov et al., Adv. Drug Deliv. Rev., 16:335-348, 1995). The probes generally have minimum fluorescence signal in their native states and become highly fluorescent after enzyme-mediated release of fluorochromes, resulting in signal amplification. Besides being useful for imaging, the new dyes can be used in a large range of biotechnological applications, such as DNA sequencing, molecular beacons and protease assays.

The chromophore attachment moiety can be any biocompatible backbone that allows one or a plurality of chromophores to be covalently linked thereto. In one embodiment, the chromophore attachment moiety is a polymer, for example, a polypeptide, a polysaccharide, a nucleic acid, or a synthetic polymer. Alternatively, the chromophore attachment moiety is a monomeric, dimeric, or oligomeric molecule. Polypeptides useful as the chromophore attachment moiety include, for example, polylysine, albumins, and antibodies. Poly(L-lysine) is a useful polypeptide chromophore attachment moiety. Other useful chromophore attachment moieties include synthetic polymers such as polyglycolic acid, polylactic acid, polyglutamic acid, poly(glycolic-co-lactic) acid, polydioxanone, polyvalerolactone, poly-ε-caprolactone, poly(3-hydroxybutyrate), poly(3-hydroxyvalerate), polytartronic acid, and poly(β-malonic acid).

Activation sites can be located in the chromophore attachment moiety, e.g., when the chromophores are linked directly to ε-amino groups of polylysine. Alternatively, each chromophore can be linked to the chromophore attachment moiety by a spacer, e.g., a spacer containing a chromophore activation site. The spacers can be oligopeptides. Oligopeptide sequences useful as spacers (or in spacers) include: Arg-Arg; Arg-Arg-Gly; Gly-Pro-Ile-Cys-Phe-Phe-Arg-Leu-Gly (SEQ ID NO:1); His-Ser-Ser-Lys-Leu-Gln-Gly (SEQ ID NO:2); Gly-Arg-Lys-Lys-Arg-Arg-Gln-Arg-Arg-Arg-Lys(FITC)-Gly-Asp-Glu-Val-Asp-Gly-Cys(QSY7)-NH₂ (SEQ ID NO:3); RRK(FITC)C-NH₂ (SEQ ID NO: 4); GRRK(FITC)C-NH₂ (SEQ ID NO:5); GRRRRK(FITC)C-NH₂ (SEQ ID NO:6); GRRGRRK(FITC)C-NH₂ (SEQ ID NO:7); GFGSVQ:FAGK(FITC)C-NH₂ (SEQ ID NO:8); GFLGGK(FITC)C-NH₂ (SEQ ID NO:9); Gly-Pro-Leu-Gly-Val-Arg-Gly-Lys(FITC)-Cys-NH₂ (SEQ ID NO: 10); Gly-D-Phe-Pip-Arg-Ser-Gly-Gly-Gly-Gly-Lys(FITC)-Cys-NH₂ (where Pip=pipecolic acid) (SEQ ID NO:11); and Gly-D-Phe-Pro-Arg-Ser-Gly-Gly-Gly-Gly-Lys(FITC)-Cys-NH₂ (SEQ ID NO: 12).

The new dyes of the invention can include one or more protective chains covalently linked to the chromophore attachment moiety. Suitable protective chains include polyhydroxyl compounds or other hydrophilic polymers such as polyethylene glycol, methoxypolyethylene glycol, methoxypolypropylene glycol, copolymers of polyethylene glycol and methoxypolypropylene glycol, polylactic-polyglycolic acid, poloxamer, polysorbate 20, dextran and its derivatives, starch and starch derivatives, and fatty acids and their derivatives. In certain embodiments of the invention, the chromophore attachment moiety is polylysine and the protective chains are methoxypolyethylene glycols.

Synthesis of NIR Fluorescence (NIRF) Dyes

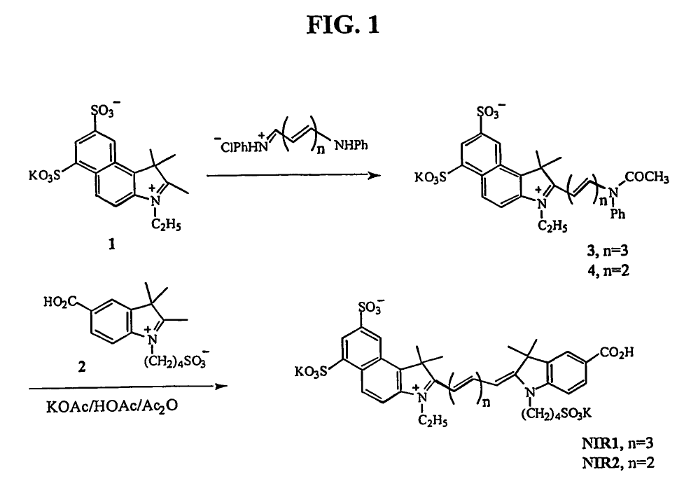

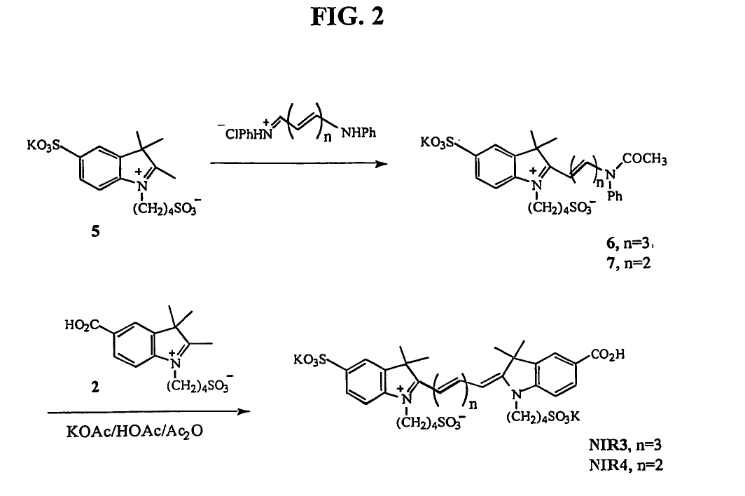

The synthetic pathways leading to eight new chromophores (referred to as NIR1, NIR2, NIR3, NIR4, NIR5, NIR6, NIR7, and NIR8) are illustrated in FIGS. 1, 2, and 3A.

The syntheses of NIR1 and NIR2 were carried out starting from 1,1,2-trimethyl-benzindoleninium-1,3-disulfonate dipotassium salt, which was converted to N-ethyl-2,3,3-trimethyl-benzindoleninium-5,7-disulfonate 1. Reaction of N-ethyl-2,3,3-trimethyl-benzindoleninium-5,7-disulfonate 1 with glutaconaldehydedianil hydrochloride and malonaldehyde dianilide hydrochloride, respectively, resulted in the intermediates 3 and 4. Intermediates 3 and 4 were stable at room temperature, even in aqueous solution, and no significant decomposition was observed over two weeks. The asymmetrical fluorochrome dyes NIR1 and NIR2 were assembled by reacting intermediates 3 and 4, respectively, with 5-carboxy-1-(4-sulfobutyl)-2,3,3-trimethyl-3H-indolenin 2.

Similarly, the syntheses of NIR3 and NIR4 began with 1-(4-sulfonatobutyl)-2,3,3-trimethylindoleninium-5-sulfonate 5, which was converted to intermediates 6 and 7 by the reaction with glutaconaldehydedianil hydrochloride and malonaldehyde dianilide hydrochloride, respectively. Like intermediates 3 and 4, intermediates 6 and 7 were also stable at room temperature. Reaction of intermediates 6 and 7 with 5-carboxy-1-(4-sulfobutyl)-2,3,3-trimethyl-3H-indolenin 2 yielded the asymmetrical fluorochrome dyes NIR3 and NIR4, respectively. The final products were >98% pure as determined by HPLC.

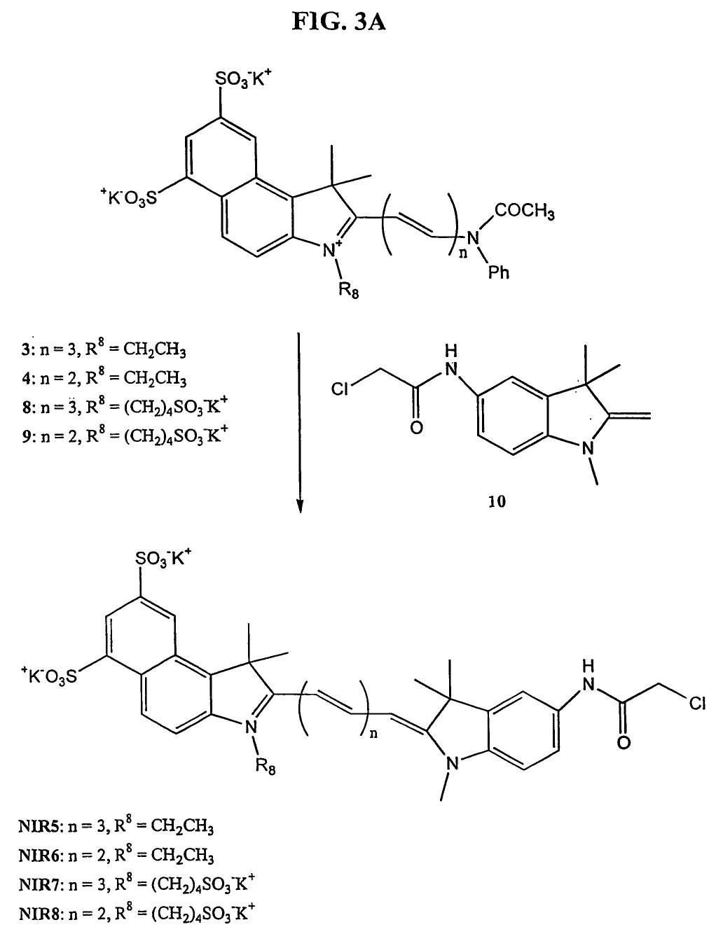

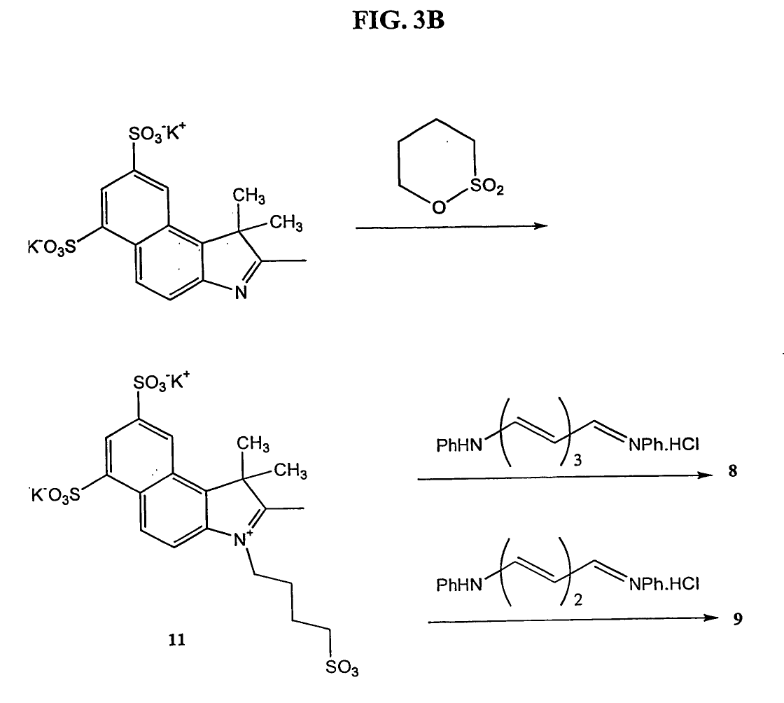

Additionally, treatment of intermediates 3, 4, 8, and 9 with 5-chloroacetamidomethyl-1,3,3-trimethyl-2methyleneindoline 10 afforded the asymmetrical fluorochrome dyes NIR5, NIR6, NIR7, and NIR8 respectively (see FIG. 3A). Intermediates 8 and 9 were prepared by the reaction between 1 11 and glutaconaldehydedianil hydrochloride and malonaldehyde dianilide hydrochloride, respectively as shown in FIG. 3B. The chloroacetamino-containing cyanines NIR5-NIR8 were purified by reversed phase semi-preparative HPLC and were found to be approximately 98% pure by reversed phase HPLC.

The synthesis of other NIRF dyes of the invention can be made reacting other indoleninium compounds with glutaconaldehydedianil hydrochloride, malonaldehyde dianilide hydrochloride, or other activated linker-forming compounds. Other activated linker-forming compounds may include those compounds that form linkers containing one or more conjugated ring structures, e.g., 12. The ring may contain e.g., four to eight members and R¹⁵ can be hydrogen, substituted or unsubstituted alkyl, halogen, or an oxygen, nitrogen, sulfur, or phosphorus containing substituent. Four and six-membered rings are preferred. For example, compound 13 can form a linker containing a six-membered ring.

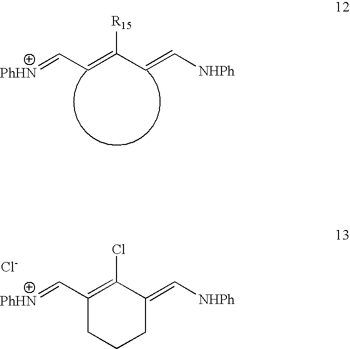

The synthesis of various cyanine-type compounds is known in the art, as described in Mishra et al., Chem. Rev., 100:1973-2011 (2000); Hamer, In The Chemistry of Heterocyclic Compounds, Weissberger, Ed., Interscience: New York, 1964, Vol. 18; VankatRaman, The Chemistry of Synthetic Dyes, Academic Press: New York, 1952, Vol. II, p. 1143; Satapathy et al., J. Ind. Chem. Soc., 45:799 (1968); Mukherjee et al., J. Ind. Chem. Soc., 47:1121 (1970); Ficken, The Chemistry of Synthetic Dyes, Vankatraman, Ed., Academic Press: New York, 1971, Vol. IV, p. 211; Gamon et al., Angew. Chem., 89:418 (1977); Dix et al., Angew. Chem., 90:8993 (1978); Mishra et al., J. Ind. Chem. Soc., 30A:886 (1991); Sahay et al., Ind. J. Chem. Soc., 27A:561 (1988); Mishra et al., Ind. J. Chem. Soc., 31B:118 (1992); and Koraiem et al., Dyes Pigments, 15:89 (1991), which are incorporated herein by reference in their entireties. Given the information herein, it is within the ability of one of ordinary skill in the art to synthesize the new chromophores without undue experimentation.

Design of the New Cyanine Dyes Certain of the new cyanine dyes of the invention bear two different heterocyclic ring systems, rendering them asymmetrical. Compounds NIR1 and NIR2 include both 3-ring and 2-ring heterocyclic systems. This design allows for fine-tuning of spectral properties by changing the substitution group on the NIR fluorochromes. The asymmetrical design can also offer improvement in the typically serious self-aggregation of large planar dyes. The latter is of particular concern, since self-aggregating fluorochromes can be poorly soluble, as is the case for indocyanine green (ICG). The new dyes's constant and large number of sulfonate groups further ensures and improves their solubility.

Enzyme Activatable Imaging Probes

We previously developed a panel of biocompatible molecular probes for the in vivo detection of specific protease activity, particularly for those proteases that play key roles in different aspects of cancer growth, metastases formation and angiogenesis (Tung et al., Canc. Res., 2000:4953-4958, 2000). We have now tested the new NIR dyes of the invention as alternative reporters in this panel. The fluorochromes were attached to a partially PEGylated graft copolymer (PGC) having a polylysine backbone as described in Bogdanov et al., Adv. Drug Deliv. Rev., 16:335-348 (1995). The probes were designed to have minimum fluorescence signal in their native states and to become highly fluorescent after enzyme mediated release of fluorochromes, resulting in signal amplification. To reduce the initial fluorescence signal, a high local concentration of fluorochromes was desired to have significant self-quenching. Since the lysine residues on the PGC were only partially PEGylated, free amino groups on the unmodified lysine side chain could be used for fluorochrome attachment. Additional free lysine residues were also needed for trypsin recognition. As a consequence, the number of fluorochromes per polymer had to be optimized to maximize the fluorescence increase after enzymatic cleavage.

For this purpose, PGC probes were labeled with different numbers of NIR2. Overall, seven conjugates were prepared, with an average of 0.2, 0.8, 1.4, 2.4, 4.3, 5.7, and 7.0 NIR2 residues per PGC molecule, respectively. The white bars in FIG. 7 represent the fluorescent signal of the labeled polymers before trypsin treatment. An increase was observed in the signal from 0.2-0.8 dye molecules/polymer, while at higher dye/polymer ratio, considerable self-quenching was observed. The black bars in FIG. 7 correspond to the fluorescence signals obtained after 3 hours of tryptic cleavage. Maximum recovery was found for 4.3 NIR2 per PGC. At this ratio, the fluorescence signal increased 5-fold in 3 hours and 9-fold in 24 hours. Interestingly, recovery was lower when more NIR2 molecules were attached to the backbone. Without wishing to be bound by theory, the observed decrease may be due to there being fewer enzyme-accessible cleavage sites on the backbone when more dye molecules are present.

Use of the New Cyanine Dyes for NIR Imaging

There are many biological processes that cannot be easily or directly monitored with MRI, PET, or CT because key molecules in these processes are not distinguishable even in the presence of currently used contrast agents. NIP technology offers unique advantages for imaging of pathology, because neither water nor many naturally occurring fluorochromes absorbs significantly in this region. Thus, NIR light penetrates tissues more efficiently than visible light or photons in the infrared region. Exogenously added contrast agents can aid in the specificity and sensitivity of disease detection. The new NIR contrast agents can be prepared in numerous forms, including as a free dye, an albumin-binding molecule, a targeting ligand, a quenched molecule, or other format.

Probe Design and Synthesis and Methods of Activation

Probe architecture, i.e., the particular arrangement of probe components, can vary, so long as the probe retains a chromophore attachment moiety, and, optionally, spacers, and one or more (e.g., a plurality) of the new chromophores linked to the chromophore attachment moiety so that the optical properties of the chromophores are altered upon activation of the imaging probe. For example, the activation sites can be in the backbone itself or in side chains. Each chromophore can be in. a separate side chain, for example, or a pair of chromophores can be in a single side chain. In the latter case, an activation site can be placed in the side chain between the pair of chromophores.

In some embodiments, the probe includes a polypeptide backbone containing only a small number of amino acids, e.g., 5 to 20 amino acids, with chromophores attached to amino acids on opposite sides of a protease cleavage (activation) site. Guidance concerning various probe components, including backbone, protective side chains, chromophores, chromophore attachment moieties, spacers, activation sites, and targeting moieties is provided in the paragraphs below.

The chromophore attachment moiety design will depend on considerations such as biocompatibility (e.g., toxicity and immunogenicity), serum half-life, useful functional groups (for conjugating chromophores, spacers, and protective groups), and cost. Useful types of chromophore attachment moieties, also referred to herein as “backbones,” include polypeptides (polyamino acids), polyethyleneamines, polysaccharides, aminated polysaccharides, aminated oligosaccharides, polyamidoamines, polyacrylic acids, and polyalcohols. In some embodiments, the backbone consists of a polypeptide formed from L-amino acids, D-amino acids, or a combination thereof. Such a polypeptide can be, e.g., a polypeptide identical or similar to a naturally occurring protein such as albumin, a homopolymer such as polylysine, or a copolymer such as a D-Tyr-D-Lys copolymer. When lysine residues are present in the backbone, the ε-amino “groups” on the side chains of the lysine residues can serve as convenient reactive groups for covalent linkage of chromophores and spacers. When the backbone is a polypeptide, the molecular weight of the probe can be from 2 kD to 1000 kD, e.g., from 4 kD to 500 kD.

The chromophore attachment moieties can also be non-covalently associated complexes, such as liposomes. Chromophores can be attached to lipids before or after liposome formation. When these complexes interact with targets, the complexes can be activated, for example, without limitation, by quenching, de-quenching, wavelength shift, fluorescence energy transfer, fluorescence lifetime change, and polarity change. The probes can be located entirely within such a liposome and released locally with disruption of the liposome (such as with acoustic resonance energy imparted at ultrasound frequencies), or can be attached at the lipid surface.

A chromophore attachment moiety can be chosen or designed to have a suitably long in vivo persistence (half-life). Alternatively, a rapidly biodegradable backbone such as polylysine can be used in combination with covalently linked protective chains. Examples of useful protective chains include polyethylene glycol (PEG), methoxypolyethylene glycol (MPEG), methoxypolypropylene glycol, polyethylene glycol-diacid, polyethylene glycol monoamine, MPEG monoamine, MPEG hydrazide, and MPEG imidazole. The protective chains can also be block-copolymers of PEG and a different polymer such as a polypeptide, polysaccharide, polyamidoamine, polyethyleneamine, or polynucleotide. Synthetic, biocompatible polymers are discussed generally in Holland et al., Advances in Pharmaceutical Sciences, 6:101-164, 1992.

A useful backbone-protective chain combination is methoxypoly(ethylene)glycol-succinyl-N-ε-poly-L-lysyine (PL-MPEG). The synthesis of this material, and other polylysine backbones with protective chains, is described in Bogdanov et al., U.S. Pat. No. 5,593,658 and Bogdanov et al., 1995, Advanced Drug Delivery Reviews, 16:335-348.

Modifications to the chromophore attachment moiety can also be made to improve delivery and activation. For example, graft copolymers can be modified to improve the probes' biological properties and/or to improve activation. For example, a 560 kD MFEG-PL graft copolymer randomly modified with Cy5.5 to yield a cathepsin B-sensitive probe (as described in the examples of U.S. Pat. No. 6,083,486) was further modified to yield a succinilated probe, i.e., the positive charges on the probe were modified to neutral or negative charges by acetylation or succinilation, respectively, which demonstrated improved activation properties.

There are numerous other chemical modifications of polymers that can be made, including changes in the charge of the polymer, changes in the polymers' hydrophobic and hydrophilic properties, changes in the size and length of the polymer side chains, and addition of attractants and/or binding moieties for enzymes. Examples of such modifications include a large number of small molecules such as succinate, acetate, amino acids, phenyl, guanidinium, tetramethylguanidinium, methyl, ethyl, propyl, isopropyl, and benzyl.

Membrane translocation signals can also be added to the imaging probes to improve deliverability. Since many graft copolymers can enter various cell types through fluid phase endocytosis, improvement of cellular uptake and assurance of cytoplasmic deposition of the imaging probe can be achieved by attaching membrane translocation (or transmembrane) signal sequences. These signal sequences can be derived from a number of sources including, without limitation, viruses and bacteria. For example, a Tat protein-derived peptide containing a caspase-3 sensitive cleavage site with the sequence—Gly-Arg-Lys-Lys-Arg-Arg-Gln-Arg-Arg-Arg-Lys(FITC)-Gly-Asp-Glu-Val-Asp-Gly-Cys(QSY7)-NH₂ —(SEQ ID NO:3) has been shown to be efficiently internalized into cells for monitoring caspase-3 activity. The sequences Gly-Arg-Lys-Lys-Arg-Gln-Arg-Arg (SEQ ID NO:15) or Gly-Arg-Lys-Lys-Arg-Arg-Gln-Arg-Arg (SEQ ID NO:16) can also be used.

Other targeting and delivery approaches can also be used such as folate-mediated targeting (Leamon & Low, 2001, Drug Discovery Today, 6:44-51), liposomes, transferrin, vitamins, carbohydrates and the use of other ligands that target internalizing receptors, including, but not limited to, somatostatin, nerve growth factor, oxytocin, bombesin, calcitonin, arginine vasopressin, angiotensin II, atrial nati-uretic peptide, insulin, glucagons, prolactin, gonadotropin, and various opioids. In addition, other ligands can be used that undergo an enzymatic conversion upon intracellular delivery that leaves the resulting conversion product trapped within the cell. Examples of such ligands include, for example, nitroheteroaromatic compounds that are irreversibly oxidized by hypoxic cells.

Intramolecular quenching by non-activated probes can occur by any of various quenching mechanisms. Several mechanisms are known, including resonance energy transfer between two chromophores. In this mechanism, the emission spectrum of a first chromophore should be very similar to the excitation of a second chromophore, which is in close proximity to the first chromophore. Efficiency of energy transfer is inversely proportional to r⁶, where r is the distance between the quenched chromophore and excited chromophore. Self-quenching can also result from chromophore aggregation or excimer formation. This effect is concentration-dependent. Quenching also can result from a nonpolar-to-polar environmental change.

To achieve intramolecular quenching, several strategies can be applied. These include: (1) linking a second chromophore, as an energy acceptor, at a suitable distance from the first chromophore; (2) linking chromophores to the backbone at high density, to induce self-quenching; and (3) linking polar chromophores in a vicinity of non-polar structural elements of the backbone and/or protective chains. Partial or full recovery of the optical properties can be obtained upon cleavage of the chromophore from neighboring chromophores and/or from a particular region, e.g., a non-polar region, of the probe.

The chromophore can be covalently linked to a chromophore attachment moiety or spacer using any suitable reactive group on the chromophore and a compatible functional group on the chromophore attachment moiety or spacer. For example, a carboxyl group (or activated ester) on a chromophore can be used to form an amide linkage with a primary amine such as the ε-amino group of the lysyl side-chain of polylysine.

In some embodiments of the invention, chromophores are linked to the chromophore attachment moiety through spacers containing activation sites. For example, oligopeptide spacers can be designed to contain amino acid sequences recognized by specific proteases associated with target tissues. Some probes of this type accumulate in tumor interstitium and inside tumor cells, e.g., by fluid phase endocytosis. By virtue of this accumulation, such probes can be used to image tumor tissues, even if the enzyme(s) activating the probe are not tumor specific.

In other embodiments of the invention, two paired chromophores in quenching positions are in a single polypeptide side chain containing an activation site between the two chromophores. Such a side chain can be synthesized as an activatable module that can be used as a probe per se, or can be linked to a backbone or targeting moiety, e.g., an albumin, antibody, receptor binding molecule, synthetic polymer, or polysaccharide. A useful conjugation strategy is to place a cysteine residue at the N-terminus or C-terminus of the molecule, and then employ SPDP for covalent linkage between the side chain of the terminal cysteine residue and a free amino group of the carrier or targeting molecule.

In other embodiments, the probes are designed to be activated by various enzymes, e.g., by cleavage. For example, prostate specific antigen (PSA), is a 33 kD chymotrypsin-like serine protease secreted exclusively by prostatic epithelial cells. Normally, this enzyme is primarily involved in post-ejaculation degradation of the major human seminal protein, and PSA concentrations are proportional to the volume of prostatic epithelium. The release of PSA from prostate tumor cells, however, is about 30-fold higher than that from normal prostate epithelium cells. Damage to basal membrane and deranged tissue architecture allow PSA to be secreted directly into the extracellular space and into the blood. Although high levels of PSA can be detected in serum, the serum PSA exists as a complex with al-antichymotrypsin protein, and is proteolytically inactive. Free, uncomplexed, activated PSA is present in the extracellular fluid from malignant prostate tissues, and PSA activity can be used as a marker for prostate tumor tissue. Moreover, prostate tumor tissue is highly enriched in PSA; therefore, spacers containing the amino acid sequence recognized by PSA can be used to produce an imaging probe that undergoes activation specifically in prostate tumor tissue. An example of a PSA-sensitive spacer is His-Ser-Ser-Lys-Leu-Gln-Gly (SEQ ID NO:2). Other PSA-sensitive spacers can be designed using information known in the art regarding the substrate specificity of PSA. See, e.g., Denmeade et al., Cancer Res. 57:49244930, 1997. These spacers can be included in the probe to make them activatable by PSA.

Another example involves cathepsin D, an abundant lysosomal aspartic protease distributed in various mammalian tissues. In most breast cancer tumors, cathepsin D is found at levels from 2-fold to 50-fold greater than levels found in fibroblasts or normal mammary gland cells. Thus, cathepsin D can be a useful marker for breast cancer. Spacers containing the amino acid sequence recognized by cathepsin D can be used to produce an imaging probe that undergoes activation specifically in breast cancer tissue. An example of a cathepsin D-sensitive spacer is the oligopeptide: Gly-Pro-Ile-Cys-Phe-Phe-Arg-Leu-Gly (SEQ ID NO:1). Other cathepsin D-sensitive spacers can be designed using information known in the art regarding the substrate specificity of cathepsin D. See, e.g., Gulnik et al., FEBS Let., 413:379-384, 1997.

Another example involves matrix metalloproteinases (MMPs). Several MMPs are expressed in cancers at much higher levels than in normal tissue and the extent of expression has been shown to be related to tumor stage, invasiveness, metastasis, and angiogenesis. MMP-2 (gelatinase) in particular, has been identified as one of the key MMPs in these processes, being capable of degrading type IV collagen, the major component of basement membranes. Based on these observations, several companies have initiated the development of different M inhibitors to treat malignancies and other diseases involving pathologic angiogenesis.

The design of proteinase inhibitors has evolved over the last decade and now largely relies on structure-based designs, the screening of combinatorial libraries, or employment of other combinatorial peptide approaches. Through these efforts, a number of broad-spectrum and more “selective” MMP inhibitors have been described and are in clinical trials, while a number of agents are in preclinical development. Efficacy testing in animals has largely been measured as suppression of tumor growth based on tumor volume measurement following treatment and by assessment of histological and anti-angiogenic effects of MMP inhibitors in human tumor xenografts. However, differences in tumor growth usually do not reach statistical significance in murine models until 10-20 days after initiation of treatment. In a clinical setting, surrogate markers of treatment efficacy such as tumor regression, time to recurrence or time to progression have been used because of the lack of more direct measures, although the limitations of such late endpoints are obvious.

MMP inhibitors can also be more effective when used in combination with chemotherapeutic agents. A specific molecular target-based pharmacodynamic assessment of each therapeutic approach would therefore be highly desirable (for estimating the relative contributions of each agent and resulting synergies). For the reasons outlined above there is a need to directly detect and monitor proteinase activities in vivo in an intact tumor environment.

Spacers containing the amino acid sequence recognized by MMP-2 can be used to produce imaging probes that undergo activation specifically in cancer tissue expressing MMP-2. An example of a MMP-2-sensitive spacer is the oligopeptide: GPLGVRGK(FITC)C—CH₂ (SEQ DNO:10). Other MMP-2-sensitive spacers can be designed using information known in the art regarding the substrate specificity of MMP-2. In addition, other MMP probes can be designed accordingly.

Various other enzymes can be exploited to provide probe activation (cleavage) in particular target tissues in particular diseases. Table 1 provides information on several exemplary enzymes and associated diseases (See Barrett et al., Handbook of Proteolytic Enzymes, Academic Press, 1998).

Protease cleavage sites can be determined and designed using information and techniques known in the art including using various compound and peptide libraries and associated screening techniques (Turk et al., Nature Biotech., 19:661-667, 2001).

In one embodiment of the present invention, when the chromophores are linked directly to the backbone, probe activation can be achieved by cleavage of the backbone. High chromophore loading of the backbone can interfere with backbone cleavage by activating enzymes such as cathepsins. Therefore, a balance between signal quenching and accessibility of the backbone by probe-activating enzymes is important. For any given backbone-chromophore combination (when activation sites are in the backbone), probes representing a range of chromophore loading densities can be produced and tested in vitro to determine the optimal chromophore loading percentage.

TABLE 1
Enzyme-Disease Associations

| Enzyme | Disease | Reference |
|---|---|---|
| Cathepsin B | Cancer, Cardiovascular | Weissleder et al., Nat. |
| | Disease, Arthritis, | Biotech., 17: 375, 1999 |
| | Neurodegenerative disease | |
| Cathepsin D | Cancer | Gulnik, FEBS Lett., 413: |
| | | 379, 1997 |
| Cathepsin K | Osteoporosis | Atley et al., Bone, 26: |
| | Bone Cancer | 241-247, 2000 |
| Cathepsin X | Cancer | Nägler et al., Biochemistry, |
| | | 38: 12648-12654, 1999 |
| Cathepsin S | Allergy, Asthma | Riese et al., J. Clin. Invest., |
| | | 101: 2351-2363, 1998 |
| Caspases | Apoptosis, Ischemia, | Xiang et al., P.N.A.S., |
| | Arthritis, Neurodegenerative | 93: 14559-14563, 1996 |
| | disease, Cardiovascular | |
| | Disease | |
| PSA | Prostate Cancer | Denmeade, Cancer Res. |
| | | 57: 4924, 1997 |
| MMP's | Cancer, Metastases, | Verheijen, Biochem. J. |
| | Inflammation, Arthritis, | 323: 603, 1997 |
| | Multiple Sclerosis, | |
| | Macular degeneration, | |
| | Cardiovascular Disease | |
| CMV | Viral | Sardana, J. Biol. Chem. |
| protease | | 269: 14337, 1994 |
| Thrombin | Blood clotting | Rijkers, Thrombosis Res., |
| | | 79: 491, 1995 |
| Beta- | Alzheimer Disease | Berezovska et al., J. Biol. |
| secretase | | Chem., 276: 30018-30023, |
| (BACE) | | 2001 |
| Urokinase | Cancer | Schmalfeldt et al., Clin. |
| plasminogen | | Cancer Res., 7: 2396, 2001 |
| activator | | |

When the chromophores are linked to the backbone through activation site-containing spacers, accessibility of the backbone by probe-activating moieties is unnecessary. Therefore, high loading of the backbone with spacers and chromophores does not significantly interfere with probe activation. For example, in such a system, every lysine residue of polylysine can carry a spacer and chromophore, and every chromophore can be released by activating enzymes.

Accumulation of a probe in a target tissue can be achieved or enhanced by binding a tissue-specific targeting moiety to the probe. The binding can be covalent or non-covalent. Examples of targeting moieties include a monoclonal antibody (or antigen-binding antibody fragment) directed against a target-specific marker, a receptor-binding polypeptide directed to a target-specific receptor, and a receptor-binding polysaccharide directed against a target-specific receptor.

Antibodies or antibody fragments can be produced and conjugated to the probes described herein using conventional antibody technology (see, e.g., Folli et al., Cancer Res., 54:2643-2649, 1994; Neri et al., Nature Biotechnology, 15:1271-1275, 1997). Similarly, receptor-binding polypeptides, such as somatostatin peptide, and receptor-binding polysaccharides can be produced and conjugated to probes of this invention using known techniques. Other targeting and delivery approaches can also be used such as folate-mediated targeting approaches (Leamon et al., Drug Discovery Today, 6:44-51, 2001), and use of liposomes, transferrin, vitamins, carbohydrates or other ligands that target internalizing receptors, including, but not limited to, nerve growth factor, oxytocin, bombesin, calcitonin, arginine vasopressin, angiotensin II, atrial nati-uretic peptide, insulin, glucagons, prolactin, gonadotropin, and various opioids. In addition, other ligands can be used that undergo an enzymatic conversion upon intracellular delivery that leaves the resulting conversion product trapped in the cell. Examples of such ligands include nitroheteroaromatic compounds that are irreversibly oxidized by hypoxic cells.

In one embodiment, activation of the imaging probe can be achieved through phosphorylation or dephosphorylation of the probe. Phosphorylation is mediated through enzymes such as kinases, which are abundantly involved in signal transduction and function by catalyzing addition of phosphate groups to serine, thronging, or tyrosine amino acids. There are a number of different types of kinases including, without limitation, receptor tyrosine kinases, the Src family of tyrosine kinases, serine/thronging kinases, and the Mitogen-Activated Protein (MAP) kinases. In addition, many of these molecules are associated with various disease states. Examples of kinases useful in the present invention and their associated diseases are listed in Table 2.

TABLE 2
Kinase - Disease Associations

| Kinase Type | Examples | Associated Diseases |
|---|---|---|
| Receptor Tyrosine Kinases | 1. Epidermal Growth Factor | 1. cancers of the digestive tract, |
| | Receptor (EGFR) | breast and colorectal cancer |
| | 2. Her2/neu | 2. breast cancer |
| | 3. Platelet-Derived Growth | 3. fibroadenomas of the breast |
| | Factor (PDGF) | |
| | 4. Vascular Endothelial | 4. angiogenesis |
| | Growth Factor (VEGF) | |
| | 5. Insulin receptor | 5. diabetes mellitus |
| Src family | 1. Lyn | 1. Wiskott-Aldrich syndrome |
| | 2. Fyn | 2. Wiskott-Aldrich syndrome |
| | 3. Bruton's Tyrosine Kinase | 3. X-Linked ammaglobulinemia |
| | (BTK) | |
| Serine/Threonine | 1. Protein Kinase C (PKC) | 1. Diabetes-mellitus-related |
| | 2. cardiovascular | 2. Alzheimer's syndrome |
| | complications | |
| Mitogen-Activated Protein | p38 | Inflammation |
| (MAP) kinases | | |

Thus, in one embodiment of the present invention, phosphorylation is used to activate the probe. The phosphorylation of the serine, thronging, or tyrosine amino acids can cause attraction of the negatively charged phosphate groups to the positively charged groups on the opposite molecule, thus bringing the chromophores into an interactive permissive position, causing changes in their optical parameters, e.g., quenching, dequenching, wavelength shift, fluorescence energy transfer, fluorescence life time change, or polarity change. The molecules can be fluorescence dyes, quenchers, and/or inducers (i.e., compounds that cause fluorescence lifetime change or polarity change). Phosphorylation can also increase the local hydrophilicity, thus decreasing the fluorescent resonance energy transfer between fluorochromes that is dependent upon local solvent concentration (e.g., resulting in decreased quenching).

In other embodiments, the probes can be activated by utilizing an enzyme that removes or modifies a functional group (e.g., a phosphate group) located on the spacer of the probe. The probe is thus designed to incorporate a target sequence or chemical structure into a spacer that is then modified or removed from the spacer to activate the probe. In one example, a phosphate-ester metabolizing enzyme such as an alkaline or acid phosphatase is used. These enzymes hydrolyze phosphate monoesters to an alcohol and an inorganic phosphate. Examples of enzymes useful in the present invention include conjugates of calf intestinal alkaline phosphatase (CIP) and PTP1B and PTEN phosphatase inhibitors, the latter two of which have been developed for diabetes and gliomas, respectively.

Other forms of chemical modification such as methylation can also be utilized to activate the probes. Methylase enzymes covalently link methyl groups to adenine or cysteine nucleotides within restriction enzyme target sequences, thus rendering them resistant to cleavage by restriction enzymes. A methylation enzyme such as S-adenosylmethionine can therefore be used to methylate a spacer of the imaging probe, thus rendering a quencher molecule resistant to restriction enzyme cleavage. Alternatively, a demethylase such as purified 5-MeC-DNA glycosylase can be used to demethylate a spacer, thus allowing restriction enzyme cleavage of a quenching molecule and the subsequent dequenching of the chromophore.

In other embodiments, probes containing mismatches or mutations in their sequence are provided wherein the function of specific DNA repair enzymes is used to activate the probe. For example, a mismatch within the spacer of the imaging probe can result in the signal being quenched. Upon the correction of this mismatch by the appropriate DNA enzyme, a conformational change occurs, allowing the dequenching of the signal. There are several enzymes involved in DNA repair, including, without limitation, poly ADP-ribose polymerase (PARP), DNA polymerases α, β, and Σ, and DNA ligase. Several human diseases result from deficiencies in DNA repair, including Ataxia-Telangiectasia, Xeroderma Pigmentosum, Cockayne Syndrome, and Santis-Caccione Syndrome. The loss of mismatch repair enzyme function has also been associated with the early development of many cancers.

Mutations can be inserted into the probe DNA in several different ways. For example, some methods of mutagenesis include: (1) use of degenerate oligonucleotides to create numerous mutations in a small DNA sequence; (2) spacer-scanning using nested deletions and complementary nucleotides to insert point mutations throughout a sequence of interest; (3) spacer-scanning using oligonucleotide-directed mutagenesis; and (4) use of the polymerase chain reaction (PCR) to generate specific point mutations.

Ubiquitin-specific target sequences can also be added to the probes, wherein the ubiquination of the target sequence allows for the chromophores to be brought into close proximity to permit energy transfer between the chromophores, thus activating the probe through any of the mechanisms listed herein. Ubiquination is an important process in the regulation of many biological processes, including angiogenesis and oxygen sensing. For example, the product of the von Hippel-Lindau (VHL) tumor suppressor gene (pVHL), whose loss of function contributes to VHL disease and also contributes to 70% of renal cell carcinomas, has been shown to directly promote degradation of Hypoxia-Indicuble-Factor (HIF) by ubiquination (Cockman et al., J., Biol. Chem., 275:25733-25741, 2000; Ohh et al., Nature Cell Biol., 2:423-427, 2000). Inhibitors of the ubiquination pathway include Lactocystin and the Calpain I inhibitor LLnL (N-acetyl-Leu-Leu-Norleucinal) (Boriello et al., Oncogene, 19(1):51-60, 2000).

In other embodiments, specific target binding sites are incorporated into the probes. These can include, without limitation, peptide substrates, enzyme binding sites, peptide sequences, sugars, RNA or DNA sequences, or other specific target binding sites or moieties. The probe is activated upon the binding of the target binding site, e.g., a change in the spectral properties of the chromophore occurs, for example, by adequate separation between the spacer and quencher. This is commonly referred to as a “molecular beacon.” Tyagi, Nature Biotech., 16:49, 2000.

A number of specific peptide substrates including cathepsin B-specific peptide substrates, MMP substrates, thrombin substrates and others are included in the probes of the present invention (see, e.g., Table 1). Examples of cathepsin B-specific substrates include RRK(FITC)C—CH₂ (SEQ ID NO:4), GRRK(FITC)C—CH₂ (SEQ ID NO:5), GRRRRK(FITC)C—CH₂ (SEQ ID NO:6), GRRGRRK(FITC)C—CH₂ (SEQ ID NO:7), GFGSVQ:FAGK(FITC)C—CH₂ (SEQ ID NO:8) (Peterson, Bioconjugate Chem., 10:553, 1999), and GFLGGK(FITC)C—CH₂ (SEQ ID NO:9), (Lu et al., Bioconjugate Chem., 12(1):129-133, 2001). An example of a MMP substrate is Gly-Pro-Leu-Gly-Val-Arg-Gly-Lys(FITC)-Cys—CH₂ (SEQ ID NO:10). Examples of thrombin-specific substrates (Rijkers D., Thrombosis Research 79:491, 1995) include Gly-D-Phe-Pip-Arg-Ser-Gly-Gly-Gly-Gly-Lys(FITC)-Cys—CH₂ (where Pip=pipecolic acid) (SEQ ID NO:11), Gly-D-Phe-Pro-Arg-Ser-Gly-Gly-Gly-Gly-Lys(FITC)-Cys—CH₂ (SEQ ID NO:12).

A monoclonal antibody (or antigen-binding antibody fragment) directed against a target-specific marker or a receptor-binding polypeptide or polysaccharide directed against a target-specific receptor can also be used to activate the probe. Specific proteins include, but are not limited to, G protein coupled receptors, nuclear hormone receptors such as estrogen receptors, and receptor tyrosine kinases.

In other embodiments, enzymes that are capable of transferring the chromophore are used to activate the probe. Specific target sequences that are recognized by enzymes involved in recombination of DNA (recombinases) are incorporated into the probe. Upon recognition of the target site by the enzyme, the chromophore is transferred to another molecule (recombination) resulting in altered spectral properties of the chromophore or removal or alteration of the quencher from the spacer. Enzymes involved in recombination are well known in the art. For example, recombinases are involved in immunoglobulin (Ig) and T cell receptor (TCR) gene rearrangements, a process involving the recombination of non-homologous gene segments, which occurs in immature B and T cells. The genes that encode these recombinases have been cloned and identified as RAG-1 and RAG-2.

The probes can also be activated by incorporating into the probe target sequences for enzymes involved in RNA splicing. This embodiment involves incorporating an RNA splicing sequence (e.g., an intron segment) on the spacer portion of the probe, resulting in the alteration of the spacer length. Activation is accomplished by changing the spectral properties of the chromophore, either by removing the quencher from the spacer of the probe, or by altering the quencher. Several methods of RNA splicing are known in the art. For example, splicing of introns from mRNA is mediated by a group of enzymes known as small nuclear RNAs (snRNAs), which complex together to form a splicosome. These enzymes splice RNA by precisely breaking sugar-phosphate bonds at the boundaries of introns and rejoining the free ends generated by intron removal into a continuous mRNA molecule. There are also alternative splicing pathways that allow for the formation of several different but related mRNAs that in turn encode different but related proteins. For example, the thyroid hormone calcitonin and the calcitonin gene-related polypeptide found in hypothalamus cells are derived from the same pre-mRNA species, but due to alternative splicing, result in two different, but related proteins.

The invention also features a fluorescent probes including a fluorochrome attachment moiety and a plurality of fluorochromes wherein the plurality of fluorochromes are chemically linked to the fluorochrome attachment moiety so that the spectral properties of the fluorochromes are altered upon “activation” of the fluorescent probe by an analyte.

An “analyte” can be a molecule or ion that binds to and activates fluorescent probes. Such analytes include, but are not limited to, H⁺, Ca²⁺, Na⁺, Mg²⁺, Mn²⁺, Cl⁻, Zn²⁺, O₂, Fe²⁺, and K⁺ions, NO, and H₂O₂.

In one embodiment of the invention, analyte binding is used to activate the probe. The binding of the analyte to the activation site causes an analyte-induced conformational change, thus bringing the fluorochromes into an interaction permissive position and causing changes in their optical parameters (e.g., quenching, dequenching, wavelength shift, fluorescence energy transfer, fluorescence life time change, or polarity change). The molecules can be fluorescent dyes, quenchers, and/or inducers (i.e., a compound that causes a fluorescence lifetime change or polarity change).

Peptides and polypeptides that selectively bind to analytes and undergo analyte-induced conformational changes are known, including peptides based on zinc finger domains and calcium-binding EF-hand domains (See, e.g., Berg and Merckle, J. Am. Chem. Soc., 111:3759-3761, 1989; Krizel (et al., Inorg. Chen., 32:937-940, 1993; Krizek and Berg, Inorg. Chem., 31:2984-2986, 1992; Kim et al., J Biol. Inorg. Chem., 6:173-81, 2001; and U.S. Pat. No. 6,197,928). A single zinc finger domain is 25-30 amino acids in length and has the consensus sequence (F/Y)-X-C-X₂₄—C-X₃-F-X₅-L-X₂-H—X₂₋₆(SEQ ID NO:13), where X is any amino acid Berg, Acc. Chem. Res., 28:14-19, 1995).

A single EF-domain is a helix-loop-helix motif that usually has 12 residues with the pattern, X-Z-X-Z-X-Z-X-Z-X-Z-Z-X (SEQ ID NO:14), where X is an amino acid that participates in metal coordination, e.g., histidine, glutamic acid, or aspartic acid, and Z represents the intervening amino acids, which can be any amino acid (bently et al., Curr. Opin. Struct. Biol., 10:637-643, 2000).

Other peptide sequences and methods to design and screen for peptides that bind to specific analytes are also known (Bar-Or et al., Eur. J. Biochenm., 268:42-47, 2001; Enzelberger et al., J. Chromatogr. A., 10:83-94, 2000; Fattorusso et al., Biopolymers, 37:401-410, 1995; Bonomo et al., Chemistry, 6:4195-4202, 2000; Ashraf et al., Bioorg. Med. Chem., 10: 1617-1620, 2000; Zoroddu et al., J. Inorg. Biochem., 84:47-54, 2001; Mukhejee et al., Indian Chem. Soc., 68:639-642, 1991; Hulsbergen et al., Recl Trav. Chim. Pays-Bas, 112:278-286, 1993; Ama et al., Bull Chem. Soc. Japan, 62:3464-3468, 1989; U.S. Pat. No. 6,083,758 and U.S. Pat. No. 5,928,955).

In another embodiment, probes can be activated by changes in H+ ion concentration or pH changes. Probes can be designed to contain spacers that are cleaved when physiological pH values are lowered. Examples of such spacers include alkylhydrazones, acylhydrazones, arylhydrazones, sulfonylhydrazones, imines, oximes, acetals, ketals, and orthoesters.

The methods of analyte activation described herein can be used to detect and/or evaluate many diseases or disease-associated conditions. The redistribution of analytes such as potassium, sodium, and calcium is often indicative of certain physiological processes and diseases including hypoxia and ischemia (e.g., cerebro-vascular ischemia due to stroke, embolism or thrombosis; ischemia of the colon; vascular ischemia due to coronary artery disease of heart disease; ischemia due to physical trauma or poisons; ischemia associated with encephalopathy; and renal ischemia). In addition, tumors are characterized by low pH values in comparison with normal tissue, as well as inflammation, particularly inflammation caused by foreign pathogens.

In another embodiment, a quencher molecule is used to quench the initial signal. Prior to activation, the quencher molecule is situated such that it quenches the optical properties of the reporter molecule (i.e., chromophore). Upon activation, the reporter molecule is de-quenched. By adopting these activated and unactivated states in a living animal or human, the reporter molecule and quencher molecule located on the probe will exhibit different signal intensities, depending on whether the probe is active or inactive. It is therefore possible to determine whether the probe is active or inactive in a living organism by identifying a change in the signal intensity of the reporter molecule, the quencher molecule, or a combination thereof. In addition, because the probe can be designed such that the quencher molecule quenches the reporter molecule when the probe is not activated, the probe can be designed such that the reporter molecule exhibits limited signal until the probe is either hybridized or digested.

There are a number of quenchers available and known to those skilled in the. art including, but not limited to, DABCYL, QSY-7 (Molecular Probes, Inc., OR), QSY-33 (Molecular Probes, Inc., OR), and fluorescence dyes such as Cy5 and Cy5.5 pare (Schobel, Bioconjugate 10:1107, 1999).

An additional method of detection includes two distinct fluorochromes (termed “fluorochrome1” and “fluorochrome2”) that are spatially near one another such that fluorescent resonance energy transfer (FRET) takes place. Thus, initially, excitation at fluorochromel's excitation wavelength results in emission at fluorochrome2's emission wavelength secondary to FRET. Activation of the probe can be determined in this embodiment as loss of signal at fluorochrome2's emission wavelength with excitation at fluorochrome1's excitation wavelength. Signal increase at fluorochromel's emission wavelength after excitation at fluorochrome1's excitation wavelength can aid the determination of activation in this case. Emission at fluorochrome2's emission wavelength after excitation at the fluorochrome2's excitation wavelength can also be used to determine local probe concentration.

Alternatively, the FRET method can be used to determine activation of probes when two components are brought into proximity after enzymatic activity (e.g., ubiquination), such that fluorochromel and fluorochrome2, which are initially spatially separated, are subsequently spatially near enough to each other for FRET to take place. Thus, activation is detected by exciting at fluorochrome1's excitation wavelength and recording at fluorochrome2's emission wavelength.

In Vitro Probe Testing

After an imaging probe is designed and synthesized, it can be tested in vitro to verify a requisite level of signal before activation. Preferably, this can be done by obtaining signal values for parameters such as quenching, de-quenching, wavelength shift, fluorescence energy transfer, fluorescence lifetime change, and polarity change of the fluorochrome-containing probe, in a dilute, physiological buffer. These values are then compared with the corresponding signal values obtained from an equimolar concentration of free chromophore in the same buffer, under the same chromophore-measuring conditions. Preferably, this comparison is done using a series of dilutions, to verify that the measurements are taking place on a linear section of the signal value vs. chromophore concentration curve.

The molar amount of a chromophore on a probe can be determined by one of ordinary skill in the art using any suitable technique. For example, the molar amount can be determined readily by near infrared absorption measurements. Alternatively, the molar amount can be determined readily by measuring the loss of reactive linking groups on the backbone or spacer, e.g., decrease in ninhydrin reactivity due to loss of amino groups.

In another procedure, the chromophore signal emittance is measured before and after treatment with an activating agent, e.g., an enzyme. If the probe has activation sites in the backbone (as opposed to in spacers), de-quenching should preferably be tested at various levels of chromophore loading. “Loading” in this context refers to the percentage of possible chromophore linkage sites on the backbone actually occupied by chromophores.

In addition, cells grown in culture can routinely be used to test the imaging probes of the present invention. Free probe molecules in cell culture medium should be non-detectable by fluorescence microscopy, while cellular uptake should result in probe activation and a fluorescence signal from probe-containing cells. Microscopy of cultured cells can thus be used to verify that activation takes place when cells take up a probe being tested. Microscopy of cells in culture is also a convenient means for determining whether activation occurs in one or more subcellular compartments.

The compositions and methods of the present invention can be used in combination with other imaging compositions and methods. For example, the methods of the present invention can be used in combination with traditional imaging modalities such as CT and MRI.

The imaging methods of the present invention can also be combined with therapeutic methods. For example, an immediate anti-tumor therapy can be employed if the probes of the present invention detect a tumor.

In Vivo Near Infrared Imaging

Although the invention involves novel imaging probes, general principles of fluorescence, optical image acquisition, and image processing can be applied in the practice of the invention. For a review of optical imaging techniques, see, e.g., Alfano et al., Ann. NY Acad. Sci., 820:248-270, 1997.

An imaging system useful in the practice of this invention typically includes three basic components: (1) a near infrared light source, (2) apparatus for separating or distinguishing emissions from light used for chromophore excitation, and (3) a detection system.

The light source provides monochromatic (or substantially monochromatic) near infrared light. The light source can be a suitably filtered white light, e.g., bandpass light from a broadband source. For example, light from a 150-watt halogen lamp can be passed through a suitable bandpass filter commercially available from Omega Optical (Brattleboro, Vt.). In some embodiments, the light source is a laser. See, e.g., Boas et al., Proc. Natl. Acad. Sci. USA 91:4887-4891, 1994; Ntziachristos et al., Proc. Natl. Acad. Sci. USA 97:2767-2772, 2000; Alexander, J. Clin. Laser Med. Surg. 9:416-418, 1991. Information on near infrared lasers for imaging can also be found on the Internet (e.g., at http://www.imds.com) and various other well-known sources.

A high pass or bandpass filter (700 nm) can be used to separate optical emissions from excitation light. A suitable high pass or bandpass filter is commercially available from Omega Optical. In the case of quantum dots, a single excitation wavelength can be used to excite multiple different fluorochromes on a single probe or multiple probes (with different activation sites), and spectral separation with a series of bandpass filters, diffraction grating, or other means can be used to independently read the different activations.

In general, the light detection system can include light-gathering/image-forming and light-detection/image-recording components. Although the light-detection system can be a single integrated device that incorporates both components, the light-gathering/image-forming and light-detection/image-recording components will be discussed separately. However, a recording device may simply record a single (time varying) scalar intensity instead of an image. For example, a catheter-based recording device can record information from multiple sites simultaneously (i.e., an image), or can report a scalar signal intensity that is correlated with location by other means (such as a radio-opaque marker at the catheter tip, viewed by fluoroscopy).

A particularly useful light-gathering/image-forming component is an endoscope. Endoscopic devices and techniques that have been used for in vivo optical imaging of numerous tissues and organs, including peritoneum (Gahlen et al., J. Photochem. Photobiol. B 52:131-135, 1999), ovarian cancer (Major et al., Gynecol. Oncol. 66:122-132, 1997), colon (Mycek et al., Gastrointest. Endosc. 48:390-394, 1998; Stepp et al., Endoscopy 30:379-386, 1998) bile ducts (Izuishi et al.,

Hepatogastroenterology 46:804-807, 1999), stomach (Abe et al., Endoscopy 32:281-286, 2000), bladder (Kriemair et al., Urol. Int. 63:27-31, 1999; Riedl et al., J., Endourol. 13:755-759, 1999), and brain (Ward, J. Laser Appl. 10:224-228, 1998) can be employed in the practice of the present invention.

Other types of light gathering components useful in the invention are catheter-based devices, including fiber optic devices. Such devices are particularly suitable for intravascular imaging. See, e.g., Tearney et al., Science 276:2037-2039, 1997; Boppart et al., Proc. Natl. Acad. Sci. USA 94:4256-4261, 1997.

Still other imaging technologies, including phased array technology (Boas et al., Proc. Natl. Acad. Sci. USA 91:48874891, 1994; Chance, Ann. NY Acad. Sci. 838:29-45, 1998), diffuse optical tomography (Cheng et al., Optics Express 3:118-123, 1998; Siegel et al., Optics Express 4:287-298, 1999), intravital microscopy (Dellian et al., Br. J. Cancer 82:1513-1518,2000; Monsky et al, Cancer Res. 59:4129-4135, 1999; Fukumura et al., Cell 94:715-725, 1998), and confocal imaging (Korlach et al., Proc. Natl. Acad. Sci. USA 96:8461-8466, 1999; Rajadhyaksha et al., J. Invest. Dermatol. 104:946-952, 1995; Gonzalez et al., J. Med. 30:337-356, 1999) can be employed in the practice of the present invention.

Any suitable light-detection/image-recording component, e.g., charge-coupled device (CCD) systems or photographic film, can be used in the invention. The choice of light-detection/image-recording component will depend on factors including type of light gathering/image forming component being used. Selecting suitable components, assembling them into a near infrared imaging system, and operating the system is within the ability of a person of ordinary skill in the art.

In some embodiments of the invention, two (or more) probes containing:
(1) chromophores that emit optical signals at different near infrared wavelengths, and
(2) activation sites recognized by different enzymes, e.g., cathepsin D and MMP2, are used simultaneously. This allows simultaneous evaluation of two (or more) biological phenomena.

In some embodiments of the invention, an additional chromophore that emits light at a different near infrared wavelength is attached to the probe that is not in an optical-quenching interaction-permissive position. Alternatively, two chemically similar probes, one activatable and one non-activatable, each labeled with a different chromophore, can be used. By using the ratio of activatable to non-activatable probe fluorescence, the activity of enzymes can be determined in a manner that is corrected for the ability of tissues to accumulate variable amounts of these probes. Both of these approaches can be used to monitor delivery of the probe, to track the probe, to calculate doses, and to serve as an internal standard for calibration purposes.

Pharmaceutically acceptable carriers, adjuvants, and vehicles can be used with the compounds of this invention. Useful carriers, adjuvants, and vehicles include, but are not limited to, ion exchangers, alumina, aluminum stearate, lecithin, serum proteins such as albumin, buffer substances such as phosphate, glycine, sorbic acid, potassium sorbate, tris(hydroxymethyl)amino methane (“TRIS”), partial glyceride mixtures of fatty acids, water, salts or electrolytes, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polypropylene block co-polymers, sugars such as glucose, and suitable cryoprotectants.

The probes of the invention can be administered in the form of a sterile injectable preparation. This preparation can be prepared by those skilled in the art of such preparations according to techniques known in the art. The possible vehicles or solvents that can be used to make injectable preparations include water, Ringer's solution, and isotonic sodium chloride solution, and 5% D-glucose solution (D5W). In addition, oils such as mono- or di-glycerides and fatty acids such as oleic acid and its derivatives can be used.

The probes of the present invention can be administered orally, parenterally, by inhalation, topically, rectally, nasally, buccally, vaginally, or via an implanted reservoir. The term “parenteral administration” includes intravenous, intramuscular, intra-articular, intrasynovial, intrasternal, intrathecal, intraperitoneal, intracisternal, intrahepatic, intralesional, and intracranial injection or infusion techniques. The probes can also be administered via catheters or through a needle to any tissue.

For ophthalmic use, the probes of the invention can be formulated as micronized suspensions in isotonic, pH-adjusted, sterile saline. Alternatively, the compositions can be formulated in ointments such as petrolatum.

For topical application, the probes can be formulated in a suitable ointment, such as petrolatum. Transdermal patches can also be used. Topical application for the lower intestinal tract or vagina can be achieved by a suppository formulation or enema formulation.

The formulation of the probe can also include an antioxidant or some other chemical compound that prevents or reduces the degradation of the baseline fluorescence, or preserves the fluorescence properties, including, but not limited to, quantum yield, fluorescence lifetime, and excitation and emission wavelengths. These antioxidants or other chemical compounds can include, but are not limited to, melatonin, dithiothreitol (dTT), defroxamine (DFX), methionine, and N-acetyl cysteine.

Dosing of the new chromophores and probes will depend on a number of factors including the instruments' sensitivity, as well as a number of subject-related variables, including animal species, age, body weight, mode of administration, sex, diet, time of administration, and rate of excretion.

Prior to use of the invention or any pharmaceutical composition of the invention, the subject can be treated with an agent or regimen to enhance the imaging process. For example, a subject can be put on a special diet prior to imaging to reduce any auto-fluorescence or interference from ingested food, such as a low pheophorbide diet to reduce interference from fluorescent pheophorbides that are derived from some foods, such as green vegetables. Alternatively, a cleansing regimen can be used prior to imaging, such as those cleansing regimens that are used prior to colonoscopies and include use of agents such as Visiciol.

The subject (patient or animal) can also be treated with pharmacological modifiers to improve image quality. For example, using low dose enzymatic inhibitors to decrease background signal relative to target signal (secondary to proportionally lowering enzymatic activity of already low-enzymatic activity normal tissues to a greater extent than enzymatically-active pathological tissues) can improve the target-to-background ratio during disease screening. As another non-limiting example, pretreatment with methotrexate to relatively increase uptake in abnormal tissue (i.e., metabolically active cancers) in conjunction with folate-based targeted delivery can be employed.

The invention is further described in the following examples, which are not intended to limit the scope of the invention described in the claims.

### EXAMPLES

### Example 1

### Synthesis and Characterization of New Chromophores NIR1-4

Fluorochrome dyes NIR1, NIR2, NIR3, and NIR4 were synthesized according to the following procedure.

Starting Materials: 1,1,2-Trimethylbenzindoleninium 1 ,3-disulfonate dipotassium salt, 5-carboxy-1-(4-sulfobutyl)-2,3,3-trimethyl-3H-indolenin 2,1-(4-sulfonatobutyl)-2,3,3-trimethylindoleninium-5-sulfonate, and 5-chloroacetamido-1,3,3-trimethyl-2-methyleneindoli 10 were synthesized according to literature methods (Mujumdar et al., Bioconjug. Chem., 7:356-362, (1996); Terpetschnig et al., Anal. Biochem., 217:197-204 (1994); Mujemdar et al., Bioconug. Chem., 4:105-111 (1993), and Gale, D. J.; Wilshire, J. F. K. J. Soc. Dyers Colour. 1974, 90, 97-100, respectively). All compounds were used in crude form.

N-ethyl-2,3,3-trimethyl-benzindoleninium-5,7-disulfonate 1: 4.7 g of 1,1,2-trimethylbenzindoleninium 1,3-disulfonate dipotassium salt 8 ml of ethyl iodide (Aldrich Chemical Co., Milwaukee, Wis.), and 50 ml of 1,2-dichlorobenzene (Aldrich) were added to a round bottom flask. The mixture was heated under an argon atmosphere at 90° C. for 12 hours and then at 125° C. for another 10 hours. After cooling the mixture to room temperature, the solvent was decanted and the solid residue was washed three times with an acetone/ether mixture. The solid was recovered by filtration and dried under vacuum to result in 4.1 g of crude N-ethyl-2,3,3-trimethyl-benzindoleninium-5,7-disulfonate 1.

Intermediate 3: 1.92 g of N-ethyl-2,3,3-trimethylbenzindoleninium-5,7-disulfonate 1 1.12 g of glutaconaldehydedianil hydrochloride (TCI America, Portland, Oreg.), 20 ml of acetic anhydride (Aldrich), and 5 ml of glacial acetic acid (Aldrich) were added to a 50 ml round bottom flask, and the resulting mixture was heated at 120° C. for 3 hours. After cooling the mixture, it was added to ethyl acetate (Aldrich), causing a solid to precipitate. The solid was recovered by filtration, and then washed twice with ethyl acetate and dried under vacuum to yield 2.2 g crude intermediate 3.

NIR1: A mixture of 0.60 g of intermediate 3, 0.33 g of 5-carboxy-1-(4-sulfobutyl)-2,3,3-trimethyl-3H-indolenin 2, 0.49 g of potassium acetate (Aldrich), 12 ml of acetic anhydride, and 5 ml of glacial acetic acid was stirred and heated at 120° C. under an argon atmosphere for 30 minutes. After cooling to room temperature, the mixture was poured into 200 ml of ethyl acetate, causing a solid to precipitate. The precipitated solid was collected by centrifugation and then dried to result in 0.86 g of crude NIR1. The crude product was further purified by reverse phase HPLC to give 14% (based on the crude product) of pure NIR1. ¹H NMR (D₂O) (400 MHz FT-NMR spectrometer): δ 1.24 (6H, s), 1.28 (3H, t), 1.54 (6H, s), 1.80 (4H, broad m), 2.90 (2H, broad t), 3.86 (2H, broad), 4.12 (2H, broad q), 5.76 (1H, broad), 6.03 (2H, broad), 7.04 (2H, broad), 7.21 (1H, d), 7.33 (1H, broad), 7.53 (1H, broad), 7.58 (1H, s), 7.65 (1H, d), 7.73 (1H, d), 8.19 (1H, s), 8.52 (1H, s), 8.71 (1H, d).

(NIR5): A mixture of 170 mg of intermediate 3, 36 mg of 5-chloroacetamido-1,3,3-trimethyl-2-methyleneindoline 10, 5 mL of acetic anhydride, 2.5 mL of glacial acid, and 140 mg of potassium acetate were stirred and heated at 115° C. under argon atmosphere for 18 minutes. After cooling to room temperature the mixture was poured into 80 mL of ethyl acetate. The precipitate was collected by centrifugation and dried to result in 120 mg of crude NIR5. The crude product was further purified by reverse phase HPLC to give 7% (based on the crude product) of pure product. ¹H NMR (D₂O/CD₃CN, 1:1): δ 1.31 (3H, t), 1.59 (6H, 6), 1.88 (6H, s), 3.47 (3H, s), 4.11 (2H, broad q), 4.16 (2H, s), 6.13-6.19 (2H, m), 6.43-6.49 (2H, broad), 7.17 (1H, d), 7.47 (2H, d), 7.65 (2H, d), 7.77-7.90 (2H, m), 8.2 (1H, s), 8.60 (1H, s), 8.82 (1H,d).

Intermediate 4: 1.70 g of N-ethyl-2,3,3-trimethylbenzindoleninium-5,7-disulfonate 1 0.93 g of malonaldehyde dianilide hydrochloride (TCI America), 20 ml of acetic anhydride, and 5 ml of glacial acetic acid were added to a 50 ml round bottom flask. The resulting mixture was then heated at 120° C. for 3 hours. Upon cooling, the mixture was poured into ethyl acetate, causing a solid to precipitate. After recovery by filtration, the solid precipitate was washed twice with ethyl acetate and dried under vacuum to yield 1.5 g crude intermediate 4.

NIR2: A mixture of 0.58 g of intermediate 4, 0.35 g of 5-carboxy-1-(4-sulfobutyl)-2,3,3-trimethyl-3H-indolenin 2, 0.49 g of potassium acetate, 12 ml of acetic anhydride, and 5 ml of glacial acetic acid was stirred and heated at 120° C. under argon atmosphere for 30 minutes. After cooling to room temperature, the mixture was poured into 200 ml of ethyl acetate, causing a solid to precipitate. The solid was collected by centrifugation and dried to yield 0.9 g of crude NIR2. The crude product was then purified by reversed phase HPLC to give 21% (based on the crude product) of pure NIR2. ¹ NMR(D₂O): δ 1.19 (3H, t), 1.24 (6H, s), 1.52 (6H, s), 1.80 (4H, broad m), 2.90 (2H, broad t), 3.90 (2H, broad m), 4.02 (2H, broad q), 5.82 (1H, broad d), 5.85 (1H, broad d), 6.14 (1H, t), 7.08 (1H, d), 7.56 (1H, s), 7.61 761-7.77 (4H, m) 8.19 (1H, s), 8.51 (1H, s), 8.70 (1H, d).

NIR6: A mixture of 121 mg of intermediate 4, 49 mg of 10, 5 mL of acetic anhydride, 2 mL of glacial acid, and 110 mg of potassium acetate were stirred and heated at 120° C. under argon atmosphere for 20 minutes. After cooling to room temperature the mixture was poured into 80 mL of ethyl acetate. The precipitate was collected by centrifugation and dried to result in 100 mg of crude NIR5. The crude product was then purified by reversed phase HPLC with a yield of 14% of pure product (based on the crude product). ¹H NMR (D2O/CD₃CN, 2:1): 67 1.24 (3H, s), 1.43 (6H, s), 1.71 (6H, s), 3.45 (3H, broad s), 3.91 (2H, s), 4.03 (2H, broad q), 5.94-6.03 (2H, broad m), 6.31 (1H, broad m), 7.11 (1H, d), 7.44 (1H, dd), 7.49 (1H, s), 7.75-7.95 (2H, m), 8.21 (1H, s), 8.55 (1H, s), 8.79 (1H, d).

Intermediate 6: In a 50 ml round flask were placed 0.80 g of 1-(4-sulfonatobutyl)-2,3,3-trimethylindoleninium-5-sulfonate 5, 0.50 g of glutaconaldehydedianil hydrochloride, 10 ml of acetic anhydride, and 5 ml of glacial acetic acid. The resulting mixture was then heated at 120° C. for 3 hours. Upon cooling, the mixture was poured into ethyl acetate, causing a solid to precipitate. After filtration, the solid precipitate was washed twice with ethyl acetate and dried under vacuum to yield 0.88 g crude intermediate 6.

NIR3: A mixture of 0.88 g of intermediate 6, 0.55 g of 5-carboxy-1-(4-sulfobutyl)-2,3,3-trimethyl-3H-indolenin 2, 0.85 g of potassium acetate, 12 ml of acetic anhydride, and 5 ml of glacial acid were stirred and heated at 120° C. under argon atmosphere for 30 minutes. After cooling to room temperature, the mixture was poured into 200 ml of ethyl acetate, causing a solid to precipitate. The precipitate was collected by centrifugation and dried to yield 0.9 g of crude NIR3. The crude product was then purified by reverse phase HPLC to give 6.7% (based on the crude product) of pure NIR3. ¹H NMR (D₂O): δ 1.36 (6H, s), 1.42 (6H, s), 1.56-1.81 (6H, broad m), 1.81-1.89 (2H, broad m), 2.84-2.90 (4H, m), 3.92 (2H, broad t), 4.06 (2H, broad t), 5.85 (1H, broad d), 6.10 (2H, broad), 6.28 (1H, broad t), 7.17 (2H, broad m), 7.26 (1H, d), 7.45 (1H, broad), 7.53 (1H, broad), 7.67-7.77 (4H, m).

Intermediate 7: 0.80 g of 1-(4sulfonatobutyl)-2,3,3-trimethylindoleninium-5-sulfonate 5, 0.45 g of malonaldehyde dianilide hydrochloride, 10 ml of acetic anhydride, and 5 ml of glacial acetic acid were added to a 50 ml round bottom flask. The resulting mixture was then heated at 120° C. for 3 hours. Upon cooling, the mixture was poured into ethyl acetate, causing a solid to precipitate. After filtration, the solid was washed twice with ethyl acetate and dried under vacuum to yield 0.74 g crude intermediate 7.

NIR4: A mixture of 0.74 g of intermediate 7, 0.45 g of 5-carboxy-1-(4-sulfobutyl)-2,3,3-trimethyl-3H-indolenin 2, 0.70 g of potassium acetate, 12 ml of acetic anhydride, and 5 ml of glacial acetic acid were stirred and heated at 120° C. under argon atmosphere for 30 minutes. After cooling to room temperature, the mixture was poured into 200 ml of ethyl acetate, causing a solid to precipitate. The solid was collected by centrifugation and dried to yield 0.9 g of crude NIR4. The crude product was then purified by reverse phase HPLC to give 8.1% (based on the crude product) of pure NIR4. ¹H NMR (D²O): 1.29 (6H, s), 1.39 (6H, s), 1.71-1.78 (6H, broad m), 1.84-1.88 (2H, broad m), 2.79-2.87 (4H, m), 3.92 (2H, broad t), 4.04 (2H, broad t). 5.82 (1H, d), 6.06 (1H, d), 6.23 (1H, t), 7.20 (1H, d), 7.25 (1 H,d) 7.60-7.75 (6H, m).

Intermediate 11: Into a 250 mL round-bottomed flask were placed 2.2 g of 1,1,2-trimethylbenzindolenium 1,3-disulfonate dipotassium salt, 1.4 mL of 1,4-butane sultone, and 20 mL of 1,2-dichlobenzene. The reaction mixture was heated under argon atmosphere at 125° C. for 24 hours. After being cooled to room temperature, the solvent was decanted and the solid was washed three times with acetone. The solid was filtered off and dried under vacuum to yield 1.82 g of crude product 11 (FIG. 3B).

Intermediate 8: In a 200 mL round-bottomed flask were placed 0.98 g of 11 (FIG. 3B), 0.56 g of glutaconaldehyde dianil hydrochloride (TCI), 10 mL of acetic anhydride, and 4 mL of acetic acid. The mixture was heated at 125° C. for 3 hours. The mixture was then precipitated from ethyl acetate upon cooling. After filtration, the solid was washed twice with ethyl acetate and dried under vacuum to yield 1.12 g of crude intermediate 8 (FIG. 3B). UV (H₂ O): 495 nm.

NIR7: A mixture of 160 mg of intermediate 8 (FIG. 3B), 34 mg of intermediate 10, 5 mL of acetic anhydride, 2.5 mL of glacial acid and 108 mg of potassium acetate were stirred and heated at 125° C. under argon atmosphere for 25 minutes. After cooling to room temperature, the mixture was poured into 80 mL of ethyl acetate. The precipitate was collected by centrifugation and dried to result in 110 mg of crude NIR7 (FIG. 3A). The crude product was then purified by reversed phase HPLC to yield 6% of pure product (based on the crude product). ¹H NMR (D)₂O/CD₃CN, 2:1): δ 1.80 (6H, s), 2.04 (6H, s), 2.09 (4H, broad m), 3.10 (3H, broad t), 3.78 (2H, broad s), 4.22 (2H, broad) 4.27 (2H, s), 6.40-6.55 (1H, broad d), 6.55-6.75 (2H,broad), 746 (1H, broad), 767 (1H, dd), 7.75-784 (1H, broad), 785, (1H, s), 7.90 (1H, d), 7.92-8.10 (2H, broad m), 8.44 (1H, s), 8.83 (1H, s), 9.02 (1H, d)

Intermediate 9: In a 200 mL round-bottomed flask were placed 0.90 g of 11 (FIG. 3B), 0.49 g of malonaldehyde dianil hydrochloride (TCI America, Portland, Oreg), 10 mL of acetic anhydride, and 4 mL of acetic acid. The mixture was heated at 120° C. for 3 hours, then precipitated from ethyl acetate upon cooling. After filtration, the solid was washed twice with ethyl acetate and dried under vacuum to yield 1.08 g of crude intermediate 9 (FIG. 3B). UV (H₂ O): 485 nm.

NIR8. A mixture of 150 mg of intermediate 9, 30 mg of intermediate 10, 5 mL of acetic anhydride, 2 mL of glacial acid, and 100 mg of potassium acetate were stirred and heated at 125° C. under argon atmosphere for 25 minutes. After cooling to room temperature the mixture was poured into 80 mL of ethyl acetate. The precipitate was collected by centrifugation and dried to result in 120 mg of crude NIR8 (FIG. 3A). The crude product was then purified by reversed phase HPLC to yield 9% of pure product (based on the crude product). ¹H NMR (D₂O/CD₃CN, 2:1): δ 1.84 (6H, s), 2.08 (6H, s), 2.10 (4H, broad m), 3.11 (3H, broad t), 3.78 (2H, broad s), 4.27 (2H, s), 4.32 (2H, broad), 6.36- (1H, broad d), 6.45 (1H, broad d), 6.64-6.71 (1H, broad m), 7.46 (1H, d), 7.68 (1H, dd), 7.84 (1H, s), 7.96 (1H, d), 8.19-8.35 (2H, broad m). 8.47 (1H, s), 8.86 (1H, s), 9.05 (1H, d)

### Example 2

### Determination of Extinction Coefficients of Fluorochrome Dyes

All of the new NIR fluorochrome dyes were purified twice by preparative HPLC, using a preparative HPLC instrument (Rainin, Woburn. Mass.) with a C18-RP preparative column (Vydec, Hesperia, Calif.) (flow rate=6 ml/min; eluant A, water with 0.1% TFA; eluant B, 90% of acetonitrile and 10% of eluant A; starting at 90% A for 5 min and then a linear gradient over 40 min to 50% A). The instrument's dual HPLC detector was set at 240 and 360 nm. The dyes were collected, and solvent was removed using a speed-vac concentrator (Savant, Holbrook, N.Y.).

The K+ions of the potassium salts were replaced with H+ to generate the corresponding free acids by ion-exchange chromatography (cation-resin, Dowex-50, 8% cross-link, 100-200 mesh).

About 20 mg of each fluorochrome dye was dissolved in 100 ml of deionized water. The absorbance was measured individually in three dilutions of the stock solution in deionized water or in 95% ethanol, using a Hitachi U-3000 spectrophotometer to determine the extinction coefficient. The fluorescence emission maxima and intensities of the dyes were obtained using a Hitachi F-4500 fluorophotometer, using dilute solutions in water and exciting at both the main absorption peak as well the short-wavelength shoulder of the main absorption peak. In the cases of NIR2, NIR4, NIR6, and NIR8, the quantum yields were calculated relative to a standard solution of the commercially available fluorochrome Cy5.5 (Amersham-Pharmacia, Piscataway, N.J.) with quantum yield of 0.29. The calculations for NIR1, NIR3, NIR5, and NIR7 were performed relative to a standard solution of another commercially available fluorochrome, Cy7 (Amersham-Pharmacia), with a quantum yield of 0.28.

### Example 3

### Activation of Fluorochrome Dyes

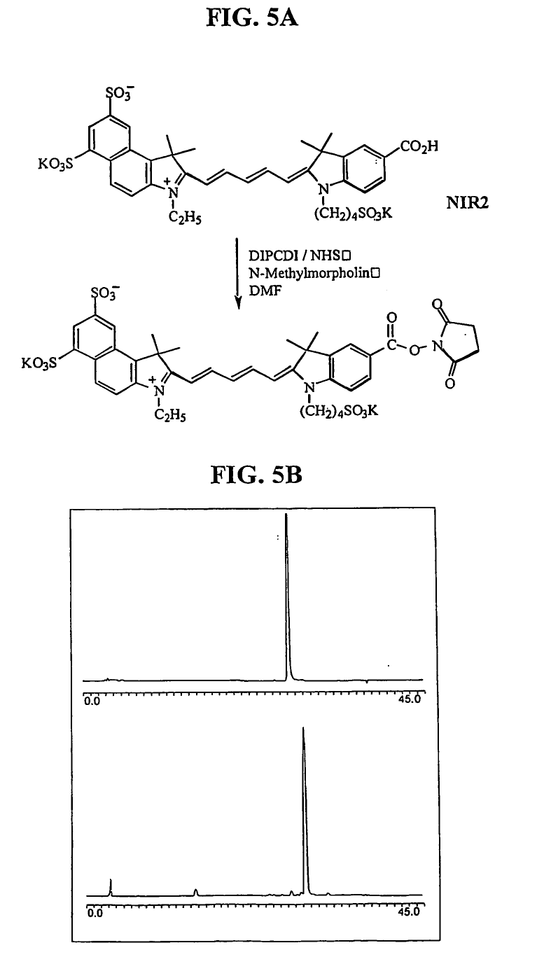

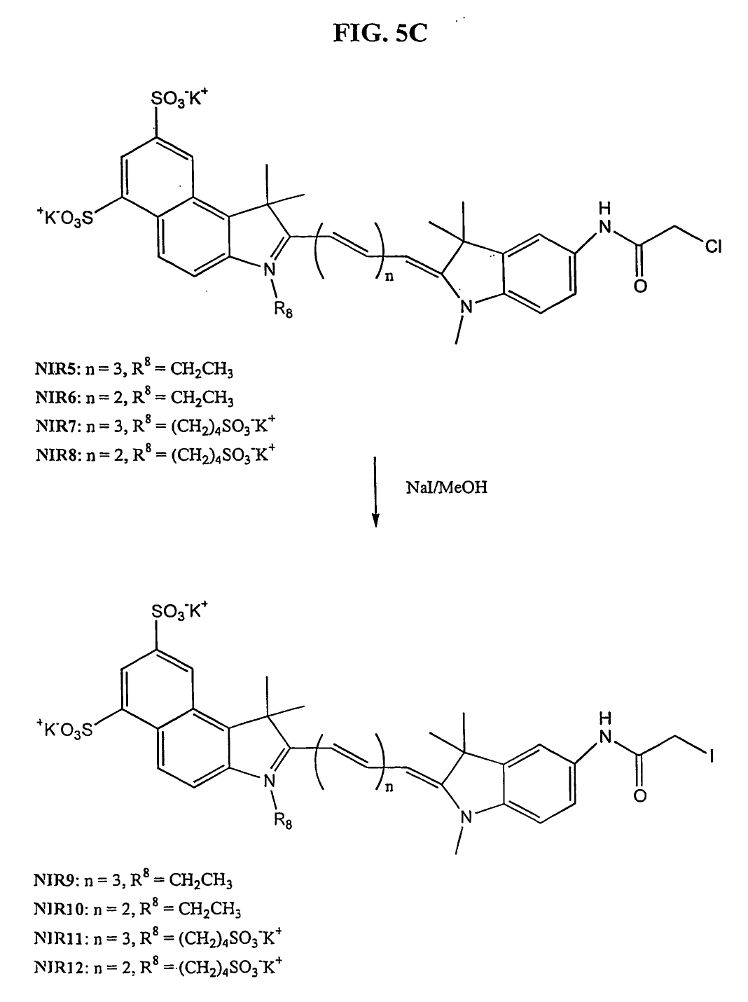

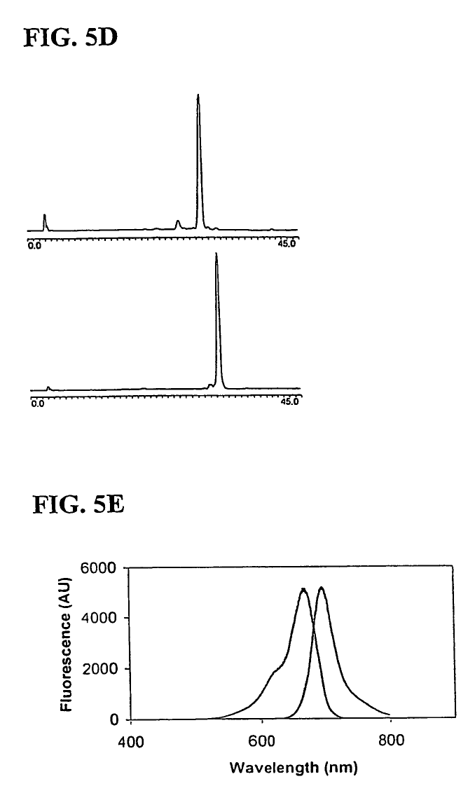

The cyanine dyes NIR1-NIR4 were converted to reactive N-succinyl esters using diisopropylcarbodiimide (DIPCDI) and N-hydroxysuccinimide in the presence of N-methylmorpholine in dimethylformamide (DMF) according to the reaction scheme shown in FIG. 5A. A nearly quantitative yield (typically >98%) was observed using reversed phase HPLC, as shown in FIG. 5B. The formation of active ester was not only confirmed by reverse phase HPLC, but also by reaction with benzylamine. FIG. 5B shows the HPLC of NIR2 (top chromatogram), as well as of its active ester (bottom chromatogram). Elution time for NIR2 and its active ester were 27.1 and 29.0 min, respectively. When the active ester reacted with benzylamine, the resultant NIR2-benzylamine conjugate showed an elution time of 32.1 min (HPLC profile not shown). The active ester was remarkably stable in water. According to PLC analysis, less than 10% of the active ester was hydrolyzed over a period of 20 days in water at 4° C.

In atypical experiment, 10 mg of dye, 30 μl of diisopropylcarbodiimide (DIPCDI; Aldrich), 50 μl of N-methylmorpholine (Aldrich), 22.0 mg of N-hydroxysuccinimide (NHS; Aldrich), and 0.5 μl of dry dimethylformamide (DMF; Aldrich) were placed in a small round bottom flask under argon atmosphere. The mixture was stirred at room temperature for 3 hours. The mixture was then poured into ether, from which a solid precipitated. After centrifugation, the ether was decanted and the remaining solid was washed four more times with ether and then dried in vacuo. According to HPLC analysis, more than 98% of the dye was converted to the corresponding active ester.

The a-chloroacetamido-containing cyanine dyes NIR5-NIR8 can be converted to the corresponding α-iodoacetamido-containing compounds. In general, the 60 -iodoacetamido functionality is a more reactive group for conjugation than the α-chloroacetamido functionality. The iodoacetamido-containing cyanine dyes NIR9-12 were obtained from NIR4-8 respectively via a halo-exchange reaction, using sodium iodide in refluxing methanol by the synthetic method shown in FIG. 5C. According to reversed-phase HPLC analysis, typically more than 98% of the chloro compound was converted to the iodo compound. The elution times for the chloro and iodo compounds are e.g., 30.0 and 31.3 min for NIR6 and NIR10 respectively.

In a typical experiment, 10 mg of 5-chloroacetamido-containing cyanine dye, 20 mg of sodium iodide, and 5 mL of methanol were placed in a small round-bottomed flask under argon atmosphere. The mixture was heated to reflux for 2.5 hours. The solvent was evaporated to afford the 5-iodoacetamido-containing cyanine dye. According to HPLC analysis, more than 98% of chloro compound was converted to the iodo compound.

The coupling of haloacetamido-containing cyanine dyes with partners containing a sulfhydryl group (—SH) was tested by the reaction of NIR10 with a cysteine containing peptide (GRRGGGGYC) (SEQ IP) NO:17). HPLC traces of NIR10 and the NIR10-peptide conjugate are shown in FIG. 5D. The elution time for the peptide conjugate was 28.8 min while that of NIR10 was 31.3 min. The structure of the NIR10-peptide conjugate was confirmed by MALDI-TOF mass spectrometry. The fluorescence excitation and emission of the NIR10-peptide conjugate are shown in FIG. 5E. The spectral properties of the NIR10-peptide conjugate were found to be similar similar to those of the free cyanine dye (Ex=666 nm; Em=695 nm). These results indicated that iodoacetamido-containing cyanine dyes have a relatively high selectivity for sulfhydryl groups and could therefore be useful for the specific labeling of sulfhydryl-containing biomolecules, e.g., proteins, peptides.

In a typical procedure, the peptide, GRRGGGGYC (SEQ ID NO:17), synthesized by standard solid phase synthesis (3.0 mg), was dissolved in 0.5 mL of 0.1 M aqueous NaHCO₃. To this solution was added 2.0 mg of NIR10 dissolved in 0.5 mL of EtOH. The mixture was stirred at RT overnight. After removal of the solvent, the NIR10-peptide conjugate was purified by reverse phase HPLC and analyzed by MALDI-TOF mass spectrometry, M+1: expected=1545, found=1548.

### Example 4

### Imaging of NIR Dyes

An NIRF reflectance imaging system as described in Mahmood et al., Radiology, 213:866-870 (1999) was used to image the new NIR dyes of the invention and to compare them to ICG. Briefly, the system included a light-tight chamber equipped with a halogen white light source and excitation bandpass filters, the first providing 610-650 nm excitation and 680-720 nm emission (“700 nm”), and the other 750-770 nm excitation and 800-820 nm emission (“800 nm”) (Omega Optical, Brattleboro, Vt.).

Equimolar NIR dyes and ICG were loaded into individual wells (0.16 nmole in 200 μl) in a clear bottom 96-well plate (Corning, Corning, N.Y.). Fluorescence was detected using a 12-bit monochrome CCD camera (Kodak, Rochester, N.Y.) equipped with a 12.5-75 mm zoom lens and emission bandpass filters at 680-720 nm or 800-820 nm (Omega Optical, Brattleboro, Vt.). Exposure time was 10 sec per image. Images were analyzed using commercially available software (Kodak Digital Science 1D software, Rochester, N.Y.).

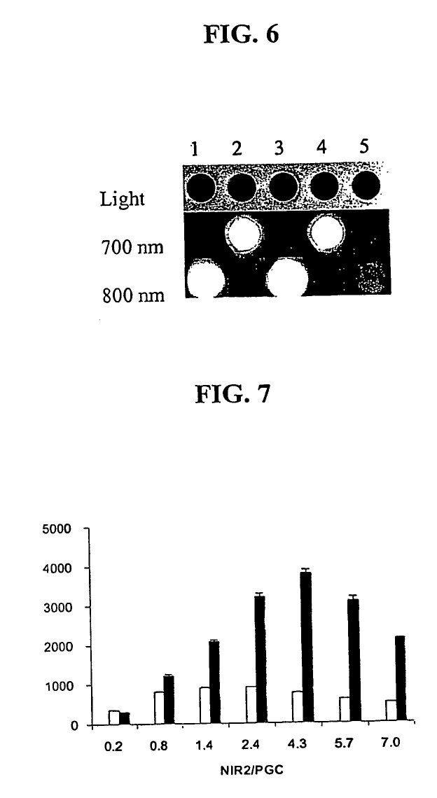

As shown in FIG. 6, the fluorescence signals of NIRs are well resolved in this fluorescence imaging system. At 700 rum, only NIR2 and NIR4 were detectable, while only NIR1 and NIR3 were detectable at 800 nm. Moreover, the NIR1 and NIR3 showed significantly better optical properties than ICG, as the signal intensities of NIR1 and NIR3 were 7- and 12-fold higher, respectively, than that of ICG.

### Example 5

### Synthesis of Enzyme-Sensitive Probe with Various Amounts of NIR2

The enzyme-sensitive probes were synthesized by reacting partially PEGylated polylysine (0.1 mg, MW=500,000 Da) with various amounts of NIR2 N-hydroxysuccinimide (NHS) ester, the concentrations of which were 0.4, 2, 4, 8, 20, 40, and 80 μM in 20 mM NaHCO₃, at room temperature for 3 hours. The NIR2-labeled polymers were then separated from excess low molecular weight reagents using a 50 kDa cutoff microconcentrator (Amicon, Beverly, Mass.). Based on NIR2 absorption measurement at 662 nm, the average numbers of NIR2 fluorochrome per PGC were 0.2, 0.8, 1.4, 2.4, 4.3, 5.7 and 7.0, respectively.

### Example 6

### Trypsin Activation of NIR2-PGC Probes

The activation of the NIRF probe was carried out in a 96-well plate with various NIR2-PGC probes. In each well, NIR2-PGC (40 pmole) in 200 μof phosphate-buffered saline (PBS) was incubated with 10 μL of trypsin solution (0.05% trypsin, 0.53 mM EDTA, Mediatech, Herndon, Va.). The reactions were monitored using a fluorescence microplate reader (Spectramax, Molecular Devices, Sunnyvale, Calif.) with excitation and emission wavelength at 662 and 684 nm, respectively. The reactions were run in duplicate.

### Example 7

### Spectral Properties of Dyes

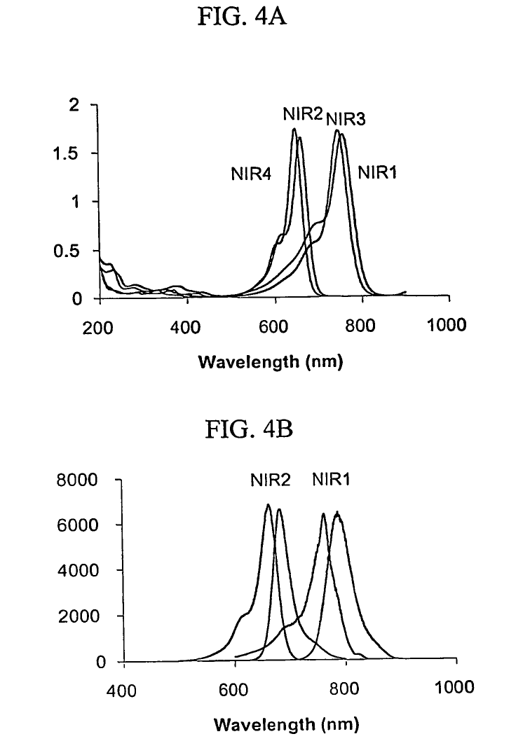

The absorption spectra of NIR1, NIR2, NIR3, and NIR4 are shown in FIG. 4A. The difference in absorbance maxima between indodicarbocyanine dye and indotricarbocyanine dye was about 100 nm. The terminal nucleus contributes very little to the absorbance maxima compared to that of the bridging methine unit. The difference in absorbance maxima between 3/2 heterocyclics (e.g., NIR2) compared to 2/2 homocycles (e.g., NIR4) was only 12 nm in water and 13 nm in ethanol. Excitation and emission spectra of NIR1 and NIR2 are shown in FIG. 4B. Indodicarbocyanine dyes NIR3 and NIR4 had a 20 nm Stokes shift of the fluorescence emission maxima, while indotricarbocyanine dyes NIR1 and NIR2 exhibited a 30 nm Stokes shift of the fluorescence maxima. The dyes had high molar extinction coefficients (ε) (i.e., above 250,000 L/mol cm¹). Quantum yields (QY) of the new fluorochromes varied from 0.23 to 0.43. Table 3 summarizes the optical properties of the compounds.

TABLE 3
Optical Properties

| | | | | Stokes | | |
|---|---|---|---|---|---|---|
| Com- | | λ_{max,abs} | λ_{max,em} | shift | ε | |
| pound | solvent | (nm) | (nm) | (nm) | (L mol⁻¹cm⁻¹⁾ | QY |
| NIR1 | water | 761 | 796 | 35 | 268,000 | 0.23 |
| | ethanol | 769 | | | | |
| NIR2 | water | 662 | 684 | 22 | 250,000 | 0.34 |
| | ethanol | 667 | | | | |
| NIR3 | water | 750 | 777 | 27 | 275,000 | 0.28 |
| | ethanol | 756 | | | | |
| NIR4 | water | 650 | 671 | 21 | 260,000 | 0.43 |
| | ethanol | 654 | | | | |

Table 4, below, summarizes the optical properties of compounds NIR9-12. These compounds were stable, and exhibited relatively high molar extinction coefficients (200,000 to 250,000) and quantum yields (0.11 to 0.24).

TABLE 4
Optical Properties of the Synthesized Sulfhydryl-Reactive
Fluorochromes

| | | | | Stokes | | |
|---|---|---|---|---|---|---|
| | | λ_{max,abs} | λ_{max,em} | Shift | ε | |
| compd | solvent | (nm) | (nm) | (nm) | L mol⁻¹cm⁻¹ | QY |
| NIR10 | H₂O/CH₃CN | 666 | 695 | 29 | 218,000 | 0.24 |
| | (1:1) | | | | | |
| NIR9 | H₂O/CH₃CN | 763 | 803 | 40 | 224,000 | 0.11 |
| | (1:1) | | | | | |
| NIR12 | H₂O/CH₃CN | 667 | 697 | 30 | 245,000 | 0.24 |
| | (2:1) | | | | | |
| NIR11 | H₂O/CH₃CN | 764 | 803 | 39 | 238,000 | 0.13 |
| | (2:1) | | | | | |

### Example 8

### Receptor-Targeted NIRF Probe

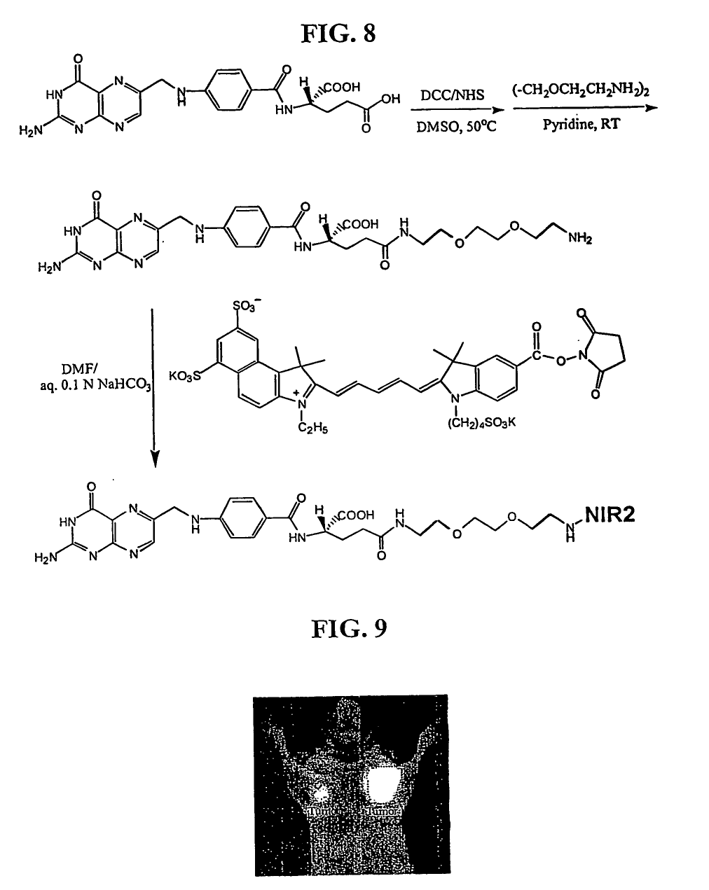

The synthetic strategy of the folate receptor-targeted probe is shown in FIG. 8. Folic acid was first synthesized as an activated ester by reacting it with N-hydroxysuccinimide (NHS) in dimethylformamide (DMF) using dicyclohexylcarbodiimide (DCC) as a condensing agent. One molar equivalent of 2,2′-(ethylenedioxy)bis-ethylamine (EDBEA) was then attached to the activated folate ester; thereafter, NIR2 was coupled to the newly generated amino group. Physical characterization indicated that the folate-NIR2 conjugate maintained all optical properties of free NIR2.

Synthesis and purification of folate-EDBEA conjugate: Into a round-bottomed flask were placed 477 mg (1 mmole) of folic acid dihydrate, 15 ml of anhydrous mixture was at heated at 50° C. for 5 hours. After cooling the mixture to room temperature, 1 ml of diisopropylamine and 1.46 ml of EDBEA (Aldrich) were added. The mixture was then stirred at room temperature for 24 hours. 20 ml of acetonitrile was then added to the mixture to precipitate the product. The product was washed three times with ethyl acetate, and then dried under vacuum. The crude product was purified using preparative HPLC (Rainin) using a C18-RP preparative column (flow rate=6 ml/minutes; eluant A, water with 0.1% TFA; eluent B, 90% of acetonitrile and 10% of eluant A; starting at 100% A for 5 minutes and then a linear gradient over 40 minutes to 60% A). The elution times for folate-EDBEA (alpha-link), and folate-EDBEA (gamma-linked) were 18.2 and 19.2 minutes, respectively. Mass spectroscopic analysis provided a mass of 573 (calcd.=571).

Synthesis and purification of folate-EDBEA-NIR2 conjugate: A solution of 3.8 mg of folate-EDBEA dissolved in 0.3 ml of 0.1 M aqueous NaHCO₃ was added to a solution of 6 mg of NIR2—CHS ester in 0.3 ml of DMF. The reaction mixture was stirred at room temperature over night in the dark. The product was then precipitated by adding the mixture to acetone. The crude product was separated from the acetone and dried. Purification of folate-EDBEA-NIR2 was carried out using the same HPLC instrument as above (flow rate=4 ml/min; eluant A, water with 0.1% TFA; eluent B, 90% of acetonitrile and 10% of eluant A; starting at 90% A for 5 minutes and then a linear gradient over 40 minutes to 50% A). The elution time for folate-EDBEA-NIR2 was 25.2 minutes. The successful conjugation of NIR2 to the folate-EDBEA was confirmed by mass spectroscopic analysis, as well as by fluorescent spectroscopy. Mass spectrum, calcd. 1401, found, 1402. Fluorescence spectroscopy showed both the fluorescence emission of NIR2 moiety (emission at 686 nm) and fluorescence emission of folate moiety (emission at 430 nm).

### Example 9

### In vivo Imaging

The free NIR2 and the folate-NIR2 compounds were both tested in tumor bearing mice. These studies were conducted to a) determine the tolerability of the agents following intravenous (IV) injection, b) tumoral enhancement as a function of time and c) differential tumor enhancement of targeted vs. non-specific probe. The study utilized folate receptor (FR) positive OVCAR-5 tumors implanted into the mammary fat pad of nude mice. All animals (n=5) tolerated the IV injection of the compounds without any signs of physiological changes over 2 weeks. Using the folate-derivatized NIR2, tumor enhancement became highly apparent within a short time after IV injection and peaked at 4 hours post-injection. A digitized photograph of one of the mice is shown in FIG. 9, illustrating that both large and small tumors can be easily detected under the NIRF imaging system in vivo. When targeting and nontargeting compounds were compared in different subsets of animals, the folate receptor targeted compound resulted in much higher tumoral fluorescence when compared to the non-targeted probes. These results indicate that NIR2 is well tolerated and is receptor-targetable.

### Example 10

### Imaging of Cell Lines

The folate derivatized NIR2 was also evaluated in a human nasopharyngeal epidermoid carcinoma, KB, cell line and a human fibrosarcoma, HT1080, cell line for its ability to improve the detection of FR positive cancers. These cell lines were selected because of putative FR overexpression (KB) or lack of detectable FR expression (HT1080) (Ross, J. F., P. K. Chaudhuri, and M. Ratnam (1994) Differential regulation of folate receptor isoforms in normal and malignant tissues in vivo and in established cell lines. Physiologic and clinical implications. Cancer, 73, 2432-43).

To confirm receptor expression levels, cellular binding/internalization was determined using ³H-folate. KB or HT1080 (10⁶ cells) grown in 12-well plates were incubated at 37° C. for different times (1, 10, 30, 60, or 120 minutes) with 50 nM ³H-folate (specific activity 34.5 Ci/mmol, American Radiolabeled Chemical Inc, St. Louis, Mo.). At the end of the incubation, cells were harvested using 0.1% Triton X-100 and the radioactivity (pmol/10⁶ cells) was determined using a scintilltion counter. For competitive inhibition studies, KB cells were incubated with different amounts of folic acid or NIR2-folate probe (5, 50, 500, and 5000 nM).

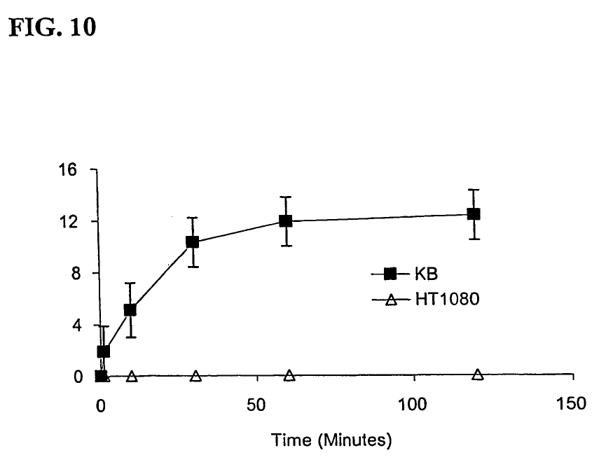

The HT1080 and KB cell lines were first characterized in terms of their putative capability of ³H-folate binding and uptake. KB and HT1080 tumor cells were incubated with ³H-folate (50 nM) up to 120 minutes. Cellular binding and uptake was quantified by scintillation counting. FIG. 10 summarizes the cellular uptake and binding data and reveals significant uptake of ³H-folate by KB cells, but essentially no uptake by HT1080 cells. For KB cells, 50% of saturation of available FR by ³H-folate was reached in 20 min and uptake reached a plateau in 60 minutes. At peak maximum 12 pmole of ³H-folate /10⁶ cells was observed under the chosen experimental conditions. In competition assays, there was a 60% decrease in bound 3H-folate in the presence of an equimolar amount (50 nM) of the free folic acid (4.97 pmole/10⁶ cells) or NIR2-folate probe (5.01 pmole/10⁶ cells). As the concentration of the free folic acid or NIR2-folate probe was increased to 5000 nM, binding of ³H-folate also decreased to 15% of its initial value, free folic acid at 1.86 pmole/10⁶ cells or NIR2-folate probe at 1.92 pmole/10⁶ cells. Competition by the NMR2-folate probe was similar to that of unconjugated folic acid. These results confirmed that fluorochrome attachment does not interfere with FR binding.

Similar to previous uptake experiments, the NIR2-folate probe was tested in cell culture using KB and HT1080 cells grown at 70% confluency on glass cover slips. The culture medium was replaced with 0.5 mL of fresh medium containing 1 μM NIR2-folate probe and incubated for 1 hour at 37° C. Cells were washed three times and fluorescence microscopy was performed using an inverted epifluorescence microscope (Zeiss Axiovert, Thornwood, N.Y.).

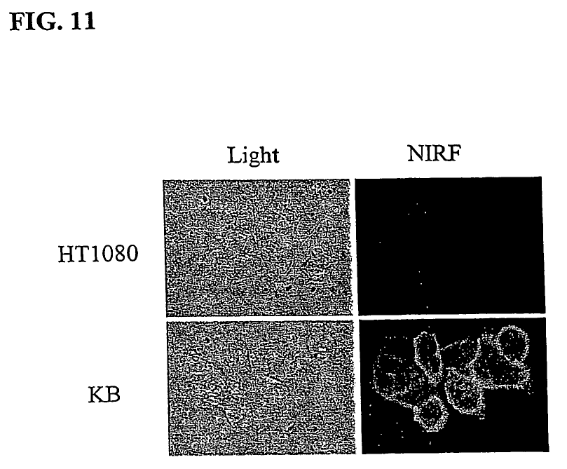

To determine the localization of fluorescent folate within cells, fluorescence microscopy was performed on KB cells incubated with the NIR-2 folate probe. As shown in FIG. 11, the KB cells showed extensive, bright fluorescence signal whereas there was essentially no binding or uptake of the NIR2-folate probe in the negative control (HT1080 cells). Fluorescence signal was seen primarily in the distribution of the plasma membrane of KB cells and in punctate vesicles in the interstitial compartment.

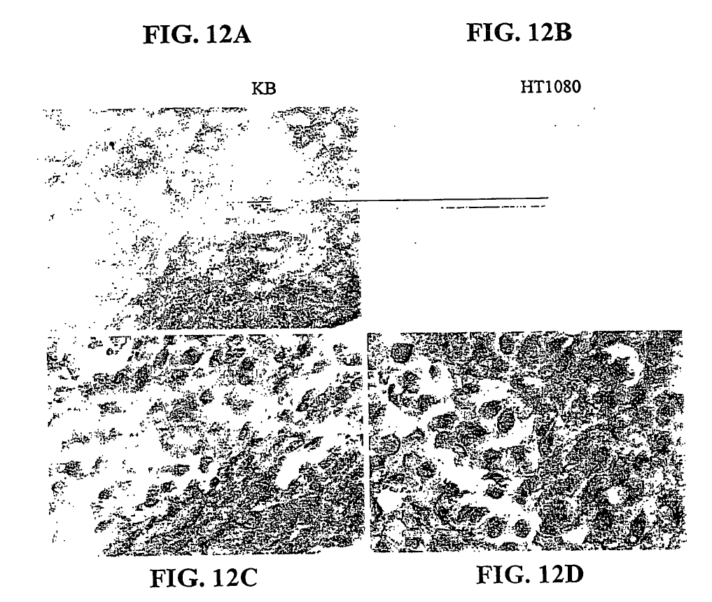

Before testing the NIR2-folate probe in vivo, tumor expression of FR was further characterized by immunohistology with FR recognizing Mab LK26. As shown in FIG. 12A, the staining showed strong immunoprecipitation in KB tumor tissues, indicating that the receptor remains overexpressed following implantation. Antibody staining showed primarily membrane and cytoplasmic staining of the KB cells. In contrast, as shown in FIG. 12B, HT1080 tumor sections were essentially negative for folate receptor. The results of Hematoxylin-eosin staining are shown in FIG. 12C (KB cells) and 12D (HT1080 cells). Hematoxylin-eosin staining revealed multiple mitotic figures present in the rapidly proliferating HT1080 fibrosarcoma, while relatively well differentiated epidermoid cells were seen in the KB tumors.

### Example 11

### Imaging Solid Tumors In Vivo

To induce solid tumors, 10⁶ KB or HT1080 cells were injected subcutaneously into mammary fat pad and the lower abdomen of 30 nude mice (average weight 20 g). Within 7-17 days after implantations, each mouse developed 3-4 tumors of 1-14 mm (mean 4.1 mm) in size. To study tumor heterogeneity, tumors with different sizes were included in the experiments. For dual-tumor experiments, six mice were injected with 10⁶ of KB and HT1080 cells on the ipsilateral and contralateral side respectively.

Thirty-six mice bearing KB and/or HT1080 tumors (n=60 each) were divided into three groups so that each group had 12 mice collectively having a total of 40 tumors; five mice collectively having a total of 18 KB tumors, five mice collectively having a total of 18 HT1080 tumors, and two mice with both KB and HT1080 tumors. Group 1 was injected with the NIR2-folate probe (2 nmole/mouse), group 2 received free NIR2 fluorochrome (not conjugated to folate, 2 nmole/mouse), and group 3 was injected with the mixture of NIR2-folate probe (2 nmole/mouse) and free folic acid (600 nmole folate/mouse). NIRF imaging was performed before and 1, 4, 24, 48 hours after tail vein injection of the probes. In two animals from each group, NIRF images were also acquired daily up to 7 days (168 hours) to study the in vivo kinetics of the probe.

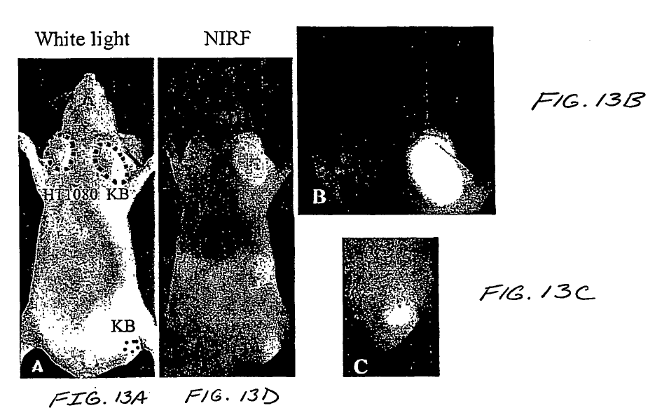

Following intravenous administration of the NIRF-folate probe, KB tumors showed significantly higher fluorescence signal intensity compared to HT1080 tumors. FIGS. 13A and 13D show the white light and NIRF images obtained 24 hours after intravenous injection of the NIR2-folate probe in a representative animal. FIG. 13B is an enlarged image of the KB and HT1080 chest tumors. The former exhibits a relatively strong fluorescence signal, while the latter does not. FIG. 13C is an enlarged image of the low abdomen tumor. The mice bearing KB tumors, tumoral fluorescence could be detected as early as 1 hour after administration of the probe (728±109 AU), which peaked at 4 hours (1210 AU±127) and then decreased (870 AU ±98 AU at 24 hours; 459 AU±48 AU at 48 hours and 255 AU±39 at 72 hours).

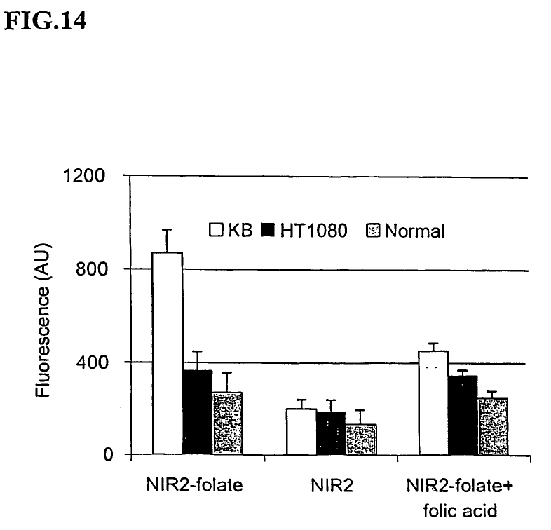

FIG. 14 is a bar graph, which shows that in tumors of equal size, there was a 2.4-fold (870 AU±98/366 AU±41, P<0.01) higher fluorescence intensity in the FR-positive KB tumors compared with the control HT1080 tumors at 24-hour images. In this set of experiments tumoral enhancement was also compared with the free NIR2 dye. At the 24 hour time point, NIR-2 fluorochrome did not result in appreciably higher signal than background. Similarly, in competition studies, fluorescence signal of FR-positive KB tumor was reduced to that of FR-negative HT1080 tumors. The competition studies indicated that the availability of free folate was able to compete off the receptor binding to NIR2-folate probe and that fluorochrome-labelled ffolic acid can still be recognized by its receptor.

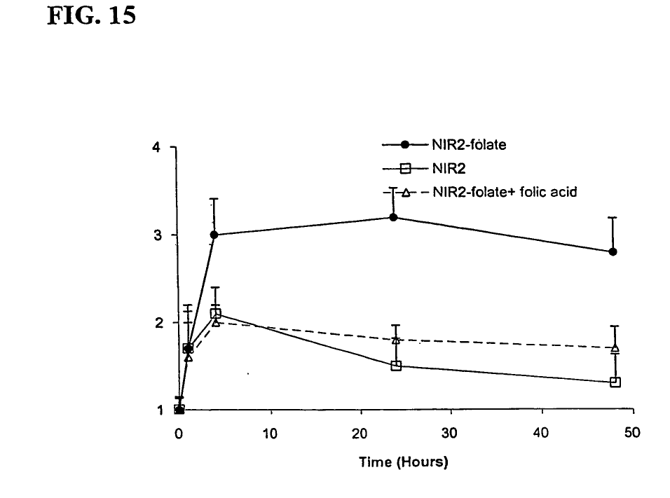

Tumor-to background contrast was measured and these ratios were plotted as a function of time after injection for the three experimental groups. The resulting graph is shown in FIG. 15. At the one hour time point, all agents had similar tumor/background ratios(y-axis) and these ratios were only moderately elevated in KB tumors. At 4 hours after injection, a significantly higher tumor/background ratio for the NIR2-folate was observed when compared to the NIR2 compound. Importantly for clinical applications, tumor/background ratios remained elevated with this probe for at least 24-48 hours indicating its potential utility for endoscopic and intraoperative use (see FIG. 15). The tumoral fluorescence signal was reduced rapidly after 72 hours (255 AU±39) and returned to the baseline (115 AU±17) in 5 days. Organ distribution of the probe was also examined after dissection. Highest fluorescence signal was observed in kidney because of high FR expression. Tumor, liver, lung and intestine were at about a similar level.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An asymmetrical chromophore compound comprising the formula: 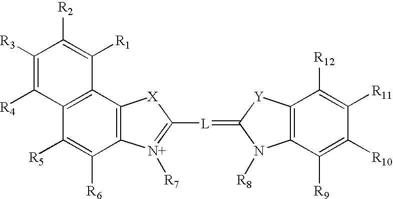
wherein L is a conjugated linker moiety;
R-₁₋₁₂ are independently selected from the group consisting of hydrogen, substituted and unsubstituted alkyl groups, substituted and unsubstituted alkenyl groups, substituted and unsubstituted alkynyl groups, substituted and unsubstituted aryl groups, sulfur-containing functional groups, phosphorus-containing functional groups, oxygen-containing functional groups, and nitrogen-containing functional groups; and
X and Y are independently selected from the group consisting of oxygen, sulfur, nitrogen, and substituted or unsubstituted methylene.

2. The compound of claim 1, wherein one or more of R₁₋₁₂ each independently comprises a reactive group for conjugation to a macromolecule.

3. The compound of claim 1, wherein one or more of R₁₋₁₂ comprise at least one substituent independently selected from the group consisting of sulfate, sulfonate, phosphate, phosphonate, halide, nitro, nitrile, and carboxylate.

4. The compound of claim 1, wherein L is (CH═CH—)CH.

5. The compound of claim 1, wherein L is (CH═CH—)₂CH.

6. The compound of claim 1, wherein L is (CH═CH—)₃CH.

7. The compound of claim 1, wherein L is (CH═CH—)₄CH.

8. The compound of claim 1, wherein L comprises one or more ring structures.

9. An asymmetrical chromophore compound comprising the formula: 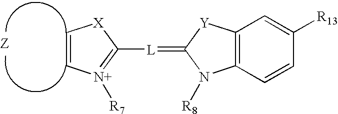
wherein L is a conjugated linker moiety;
R₇ and R₈ are independently selected from the group consisting of hydrogen, substituted and unsubstituted alkyl groups, substituted and unsubstituted alkenyl groups, substituted and unsubstituted alkynyl groups, substituted and unsubstituted aryl groups, sulfur-containing functional groups, phosphorus-containing functional groups, oxygen-containing functional groups, and nitrogen-containing functional groups; and;
X and Y are independently selected from the group consisting of oxygen, sulfur, and substituted or unsubstituted methylene;
Z is a group of nonmetallic atoms necessary for forming a substituted or unsubstituted, condensed aromatic ring or ring system;
R₁₃ is C(O)OR₁₄ or NHC(O)CH₂J;
R₁₄ is H or 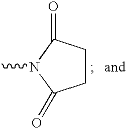
J is halo.

10. The compound of claim 9, wherein R₁₄ is H.

11. The compound of claim 9, wherein J is Cl or I.

12. An asymmetrical chromophore compound comprising the formula: 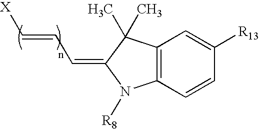
wherein X is selected from the group consisting of: 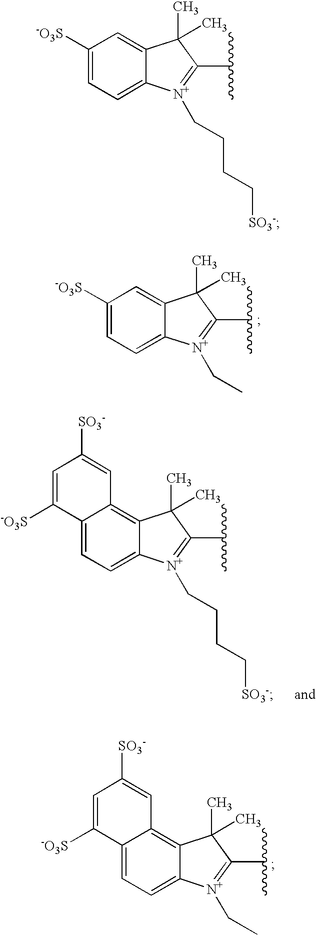
wherein,
n=2or3;
R₈ is selected from the group consisting of hydrogen, substituted and unsubstituted alkyl groups, substituted and unsubstituted alkenyl groups, substituted and unsubstituted alkynyl groups, substituted and unsubstituted aryl groups, sulfur-containing functional groups, phosphorus-containing functional groups, oxygen-containing functional groups, and nitrogen-containing functional groups;
R₁₃ is C(O)OR₁₄ or NHC(O)CH₂J;
R₁₄ is H or 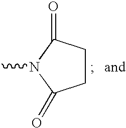
J is halo.

13. The compound of claim 12, wherein R₈ is CH₃ or (CH₂)₄SO₃⁻.

14. The compound of claim 12, wherein R₁₄ is H.

15. The compound of claim 12, wherein J is Cl or I.

16. An in vivo method of imaging a tissue in a subject, the method comprising:
a) conjugating to a targeting ligand a chromophore of claims 1 or 9;
b) combining the conjugated chromophore with an excipient to form an administerable formulation;
c) administering the formulation to the tissue; and
d) detecting the conjugated chromophore in the tissue to provide a fluorescence image of the tissue.

17. The method of claim 16, wherein the targeting ligand is a receptor binding ligand.

18. The method of claim 16, wherein the tissue is a tumor tissue.

19. The method of claim 16, wherein the subject is mammal.

20. The method of claim 16, wherein the subject is a human.

21. An in vitro method of imaging a tissue, the method comprising:
a) conjugating to a targeting ligand a chromophore of claims 1 or 9;
b) contacting the conjugated chromophore with the tissue; and
c) detecting the conjugated chromophore in the tissue to provide a fluorescence image of the tissue.
